(19) United States
(12) Patent Application Publication
(10) Pub. No.: US 2018/0310511 A1
(43) Pub. Date: Nov. 1, 2018

(54) PEPPER VARIETY NUN 70042 PPH

(71) Applicant: NUNHEMS B.V., Nunhem (NL)

(72) Inventor: Jesus Saldivar DAVILA, Nunhem (NL)

(21) Appl. No.: 15/966,979

(22) Filed: Apr. 30, 2018

Related U.S. Application Data
(62) Division of application No. 15/440,672, filed on Feb. 23, 2017.
(60) Provisional application No. 62/404,291, filed on Oct. 5, 2016.

(51) Int. Cl. A01H 5/08

(52) U.S. Cl. CPC A01H 5/08

(57) ABSTRACT

The invention provides a new and distinct hybrid variety of pepper, NUN 70039 PPH or NUN 70041 PPH or NUN 70042 PPH.

### CROSS REFERENCE TO RELATED APPLICATIONS

This application claims the benefit of U.S. Provisional Application Ser. No. 62/404,291, filed Oct. 5, 2016, the contents of which are herein incorporated by reference in their entirety.

### FIELD OF THE INVENTION

The present invention relates to the field of plant breeding and, more specifically, to the development of pepper varieties NUN 70039 PPH (also designated as NUN 70039 or NUN70039 F1 or NUN 70039 hybrid), NUN 70041 PPH (also designated as NUN 70041 or NUN 70041 F1 or NUN 70041 hybrid) and NUN 70042 PPH (also designated as NUN 70042 or NUN 70042 F1 or NUN 70042 hybrid). The invention further relates to vegetative reproductions of NUN 70039 PPH, NUN 70041 PPH and NUN 70042 PPH, methods for in vitro tissue culture of NUN 70039 PPH, NUN 70041 PPH and NUN 70042 PPH, an explant and also to phenotypic variants of NUN 70039 PPH, NUN 70041 PPH and NUN 70042 PPH.

The goal of vegetable breeding is to combine various desirable traits in a single variety/hybrid. Such desirable traits may include greater yield, resistance to diseases, insects or other pests, tolerance to heat and drought, better agronomic quality, higher nutritional value, enhanced growth rate and improved fruit properties.

Breeding techniques take advantage of a plant's method of pollination. There are two general methods of pollination: a plant self-pollinates if pollen from one flower is transferred to the same or another flower of the same genotype. A plant cross-pollinates if pollen comes to it from a flower of a different genotype.

Plants that have been self-pollinated and selected for (uniform) type over many generations become homozygous at almost all gene loci and produce a uniform population of true breeding progeny of homozygous plants. A cross between two such homozygous plants of different lines produces a uniform population of hybrid plants that are heterozygous for many gene loci. The extent of heterozygosity in the hybrid is a function of the genetic distance between the parents. Conversely, a cross of two plants each heterozygous at a number of loci produces a segregating population of hybrid plants that differ genetically and are not uniform. The resulting non-uniformity makes performance unpredictable.

The development of uniform varieties requires the development of homozygous inbred plants, the crossing of these inbred plants, and the evaluation of the crosses. Pedigree breeding and recurrent selection are examples of breeding methods that have been used to develop inbred plants from breeding populations. Those breeding methods combine the genetic backgrounds from two or more plants or various other broad-based sources into breeding pools from which new lines are developed by selfing and selection of desired phenotypes. The new lines are evaluated to determine which of those have commercial potential. One crop species which has been subject to such breeding programs and is of particular value is the pepper.

One crop species which has been subject to such breeding programs and is of particular value is the pepper. Pepper (Capsicum spp.) is naturally a diploid and the basic chromosome number of the genus is x=12, most are 2n=2x=24, including the cultivated ones. A few wild species have 2n=26. Ploidy changes (both tetraploidy and haploidy) are relatively easy to induce in Capsicum species. Doubled haploids have proved particularly valuable in the analysis of the genetically complex basis of some resistances to pests and diseases.

The genus Capsicum originated in American tropics. The fruit of most species of Capsicum produce a strong burning sensation (pungency or spiciness) in the mouth of the unaccustomed eater due to the presence of capsaicin (methyl vanillyl nonenamide), a lipophilic chemical. Capsaicin can be present in large quantities in the placental tissue (which holds the seeds), the internal membranes, and to a lesser extent, the other fleshy parts of the fruits of plants in this genus. The seeds themselves do not produce any capsaicin. The amount of capsaicin in the fruit is highly variable and dependent on genetics and environment, giving almost all types of Capsicum varied amounts of perceived heat. The most recognizable Capsicum without capsaicin is the bell pepper, a cultivar of Capsicum annuum, which has a zero rating on the Scoville scale. The lack of capsaicin in bell peppers is due to a recessive gene that eliminates capsaicin and, consequently, the “hot” taste usually associated with the rest of the Capsicum family.

Many of the peppers currently used which are used in the fresh of processed marked in the United States are seeded hybrid varieties. Hybrid varieties offer the advantages of easy combination of dominant and recessive traits, such as disease resistance, from a set of inbred parents, as well as careful control of parentage.

Many different pepper cultivars have been produced, and pepper breeding efforts have been underway in many parts of the world. Some breeding objectives include varying the color, texture and flavor of the fruit. Other objectives include disease or pest resistance, optimizing flesh thickness, yield, suitability to various climatic circumstances, heat, solid content (% dry matter), and sugar content.

Advances in biotechnology have also resulted in genetically engineered pepper plants with improved traits. For example, fungal resistant pepper plants comprising a PepEST or PepDef gene where the expression of the nucleic acid sequence in the plant resulted in increased resistance to fungal infection (see e.g. US application 20060037100, Feb. 16, 2006).

While breeding efforts to date have provided a number of useful pepper varieties with beneficial traits, there remains a great need in the art for new varieties with further improved traits. Such plants would benefit farmers and consumers alike by improving crop yields and/or quality.

### SUMMARY OF THE INVENTION

In one aspect of the invention, a seed of pepper variety NUN 70039 PPH is provided, wherein a representative sample of said seed has been deposited under Accession Number NCIMB ______. In another aspect of the invention, a seed of pepper variety NUN 70041 PPH is provided, wherein a representative sample of said seed has been deposited under Accession Number NCIMB ______. In also another aspect of the invention, a seed of pepper variety NUN 70042 PPH is provided, wherein a representative sample of said seed has been deposited under Accession Number NCIMB ______. The pepper seed of any one of the varieties of the invention may be provided as an essentially homogeneous population of pepper seed. Therefore, seed of any one of the varieties of the invention may be defined as forming at least about 97% of the total seed, including at least about 98%, 99% or more of the seed. The population of pepper seed may be particularly defined as being essentially free from other seed. The seed population of any one of the varieties may be separately grown to provide an essentially homogeneous population of pepper plants according to the invention. Also encompassed are a plant grown from a seed of pepper variety NUN 70039 PPH, NUN 70041 PPH and NUN 70042 PPH and a plant part thereof.

In another aspect the invention provides for a hybrid variety of C. annuum called NUN 70039 PPH or NUN 70041 PPH or NUN 70042 PPH. The invention also provides for a seed or a plurality of seeds of any one of the varieties, a plant produced from growing the seed of the new variety NUN 70039 PPH or NUN 70041 PPH or NUN 70042 PPH, and a progeny of any of these. Especially, a progeny retaining all or all but one, two or three of the “distinguishing characteristics” or all or all but one, two or three of the “morphological and physiological characteristics” or essentially all physiological and morphological characteristics of NUN 70039 PPH or NUN 70041 PPH or NUN 70042 PPH referred to herein, is encompassed herein as well as methods for producing these.

In one aspect, such progeny have all the physiological and morphological characteristics of pepper variety NUN 70039 PPH or NUN 70041 PPH or NUN 70042 PPH when grown under the same environmental conditions. In another aspect such progeny have all or all but one, two or three of the physiological and morphological characteristics listed in Table 1 and/or 2 for pepper variety NUN 70039 PPH or NUN 70041 PPH or NUN 70042 PPH when measured under the same environmental conditions (i.e. evaluated at significance levels of 1%, 5% or 10% significance, which can also be expressed as a p value).

In another aspect a plant of the invention or said progeny plants of NUN 70039 PPH has/have 3, 4, 5, 6, 7, 8, or more or all of the distinguishing characteristics: 1) average fruit flesh thickness; 2) average fruit calyx diameter; 3) average pedicel length; 4) presence of fruits with 2 locules; 5) average plant width; 6) average fruit placenta length; 7) average number of seeds per fruit; 8) average leaf petiole length; and 9) average number of calyx lobes, in addition to 3, 4, 5, 6, 7, 8, or more, or all of the other (average) characteristics as listed in Table 1 and/or 2. NUN 70039 PPH is a jalapeno hot pepper.

In another aspect a plant of the invention or said progeny plants of NUN 70041 PPH has/have 3, 4, 5, 6, 7, 8, or more or all of the distinguishing characteristics: 1) average fruit length; 2) average fruit flesh thickness at mid-point; 3) average number of seeds per fruit; 4) average pedicel length; 5) average flower diameter; 6) average leaf length; 7) average leaf petiole length; 8) typical leaf blistering; 9) average petiole diameter; and 10) absence of fruit with five or more locules, in addition to 3, 4, 5, 6, 7, 8, or more, or all of the other (average) characteristics as listed in Table 1 and/or 2. NUN 70041 PPH is a jalapeno hot pepper.

In another aspect a plant of the invention or said progeny plants of NUN 70042 PPH has/have 3, 4, 5, 6, 7, 8, or more or all of the distinguishing characteristics: 1) average fruit length; 2) average fruit width; 3) typical fruit apex shape; 4) average pedicel thickness; 5) average fruit seed cavity length; 6) average fruit seed cavity diameter; 7) typical leaf blistering; 8) typical style length; 9) average leaf length; and 10) average plant width, in addition to 3, 4, 5, 6, 7, 8, or more, or all of the other (average) characteristics as listed in Table 1 and/or 2. NUN 70042 PPH is a jalapeno hot pepper.

Further, a pepper fruit produced on a plant grown from seed of NUN 70039 PPH or NUN 70041 PPH or NUN 70042 PPH is provided.

In yet another embodiment of the invention, a plant is provided having one, two or three physiological and/or morphological characteristics which are different from those of NUN 70039 PPH or NUN 70041 PPH or NUN 70042 PPH and which otherwise has all the physiological and morphological characteristics of NUN 70039 PPH or NUN 70041 PPH or NUN 70042 PPH as listed in Table 1 and/or 2, wherein a representative sample of seed of variety NUN 70039 PPH has been deposited under Accession Number NCIMB ______, a representative sample of seed of variety NUN 70041 PPH has been deposited under Accession Number NCIMB ______, and a representative sample of seed of variety NUN 70042 PPH has been deposited under Accession Number NCIMB ______.

Further, a vegetatively propagated plant of variety NUN 70039 PPH or NUN 70041 PPH or NUN 70042 PPH, or a part thereof, is provided having all or all but one, two or three of the morphological and physiological characteristics of NUN 70039 PPH or NUN 70041 PPH or NUN 70042 PPH when grown under the same environmental conditions.

Also a plant part derived from variety NUN 70039 PPH or NUN 70041 PPH or NUN 70042 PPH is provided, wherein said plant part is selected from the group consisting of: a fruit, a harvested fruit, a part of a fruit, a leaf, a part of a leaf, pollen, an ovule, a cell, a petiole, a shoot or a part thereof, a stem or a part thereof, a root or a part thereof, a root tip, a cutting, a seed, a part of a seed, seedcoat or another maternal tissue which is part of a seed grown on NUN 70039 PPH or NUN 70041 PPH or NUN 70042 PPH, hypocotyl, cotyledon, a scion, a stock, a rootstock, a pistil, an anther, and a flower or a part thereof. Fruits are particularly important plant parts. In yet another aspect, a seed of NUN 70039 PPH or NUN 70041 PPH or NUN 70042 PPH is provided. In still another aspect, a seed growing or grown on a plant of NUN 70039 PPH or NUN 70041 PPH or NUN 70042 PPH is provided.

### Definitions

“Pepper” refers herein to plants of the species Capsicum annuum or frutescens, and fruits thereof. The most commonly eaten part of a pepper is the fruit or berry. The fruit comprises a stem or peduncle or pedicel, calyx, placenta, fruit wall, veins, shoulder, base, apex, locule or lobe, septa, exocarp, mesocarp, endocarp, pericarp, optionally secondary peppers, optionally capsaicin glands and optionally seed. The stem or peduncle or pedicel, calyx, placenta, fruit wall, veins, shoulder, base, apex, locule or lobe, septa, exocarp, mesocarp, endocarp, pericarp, secondary peppers, capsaicin glands and seedcoat of the seed are maternal tissues, that is they are genetically identical to the plant on which they grow.

“Cultivated pepper” refers to plants of Capsicum annuum, or a closely related species, i.e. varieties, breeding lines or cultivars of the species C. annuum as well as crossbreds thereof, or crossbreds with other Capsicum species, cultivated by humans and having good agronomic characteristics; preferably such plants are not “wild plants”, i.e. plants which generally have much poorer yields and poorer agronomic characteristics than cultivated plants and e.g. grow naturally in wild populations. “Wild plants” include for example ecotypes, PI (Plant Introduction) lines, landraces or wild accessions or wild relatives of Capsicum and related species.

The terms “pepper plant designated NUN 70039 PPH”, “NUN 70039”, “70039 PPH”, “NUN 70039 PP”, “NUN 70039 F1”, “NUN 70039 hybrid” or “variety designated 70039 PPH” are used interchangeably herein and refer to a pepper plant of variety NUN 70039 PPH, representative seed of which having been deposited under Accession Number NCIMB ______.

The terms “pepper plant designated NUN 70041 PPH”, “NUN 70041”, “70041 PPH”, “NUN 70041 pp”, “NUN 70041 F1”, “NUN 70041 hybrid” or “variety designated 70041 PPH” are used interchangeably herein and refer to a pepper plant of variety NUN 70041 PPH, representative seed of which having been deposited under Accession Number NCIMB ______.

The terms “pepper plant designated NUN 70042 PPH”, “NUN 70042”, “70042 PPH”, “NUN 70042 PP”, “NUN 70042 F1”, “NUN 70042 hybrid” or “variety designated 70042 PPH” are used interchangeably herein and refer to a pepper plant of variety NUN 70042 PPH, representative seed of which having been deposited under Accession Number NCIMB ______.

A “seed of NUN 70039 PPH” refers to an F1 hybrid seed represented by the deposit with Accession Number NCIMB ______. It contains an embryo of NUN 70039 PPH, or a “F1 hybrid embryo”. When said seed is planted, it grows into a plant of NUN 70039 PPH.

A “seed of NUN 70041 PPH” refers to an F1 hybrid seed represented by the deposit with Accession Number NCIMB ______. It contains an embryo of NUN 70041 PPH, or a “F1 hybrid embryo”. When said seed is planted, it grows into a plant of NUN 70041 PPH.

A “seed of NUN 70042 PPH” refers to an F1 hybrid seed represented by the deposit with Accession Number NCIMB ______. It contains an embryo of NUN 70042 PPH, or a “F1 hybrid embryo”. When said seed is planted, it grows into a plant of NUN 70042 PPH.

A “seed grown on NUN 70039 PPH” refers to a seed grown on a mature plant of NUN 70039 PPH or inside a fruit of NUN 70039 PPH. The “seed grown on NUN 70039 PPH” contains tissues and DNA of the maternal parent, NUN 70039 PPH. The “seed grown on NUN 70039 PPH” contains an F2 embryo. When said seed is planted, it grows into a first generation progeny plant of NUN 70039 PPH.

A “seed grown on NUN 70041 PPH” refers to a seed grown on a mature plant of NUN 70041 PPH or inside a fruit of NUN 70041 PPH. The “seed grown on NUN 70041 PPH” contains tissues and DNA of the maternal parent, NUN 70041 PPH. The “seed grown on NUN 70041 PPH” contains an F2 embryo. When said seed is planted, it grows into a first generation progeny plant of NUN 70041 PPH.

A “seed grown on NUN 70042 PPH” refers to a seed grown on a mature plant of NUN 70042 PPH or inside a fruit of NUN 70042 PPH. The “seed grown on NUN 70042 PPH” contains tissues and DNA of the maternal parent, NUN 70042 PPH. The “seed grown on NUN 70042 PPH” contains an F2 embryo. When said seed is planted, it grows into a first generation progeny plant of NUN 70042 PPH.

A “fruit of NUN 70039 PPH” refers to a pepper fruit containing maternal tissues of NUN 70039 PPH as deposited under Accession Number NCIMB ______. In one option, the fruit contains seed grown on NUN 70039 PPH. In another option, the fruit does not contain seed, that is the fruit is parthenocarpic. The skilled person is familiar with methods for inducing parthenocarpy. Those methods comprise chemically or genetically inducing parthenocarpy. Compounds suitable for chemically inducing parthenocarpy include auxins, gibberellins and cytokinins. Genetic parthenocarpy can a.o. be induced by CaARF8 mutants (Tiwari et al., BMC Plant Biology 201111:143 DOI: 10.1186/1471-2229-11-143) or as in WO 2013078319.

A “fruit of NUN 70041 PPH” refers to a pepper fruit containing maternal tissues of NUN 70041 PPH as deposited under Accession Number NCIMB ______. In one option, the fruit contains seed grown on NUN 70041 PPH. In another option, the fruit does not contain seed, that is the fruit is parthenocarpic. The skilled person is familiar with methods for inducing parthenocarpy. Those methods comprise chemically or genetically inducing parthenocarpy. Compounds suitable for chemically inducing parthenocarpy include auxins, gibberellins and cytokinins. Genetic parthenocarpy can a.o. be induced by CaARF8 mutants (Tiwari et al., BMC Plant Biology 201111:143 DOI: 10.1186/1471-2229-11-143) or as in WO 2013078319.

A “fruit of NUN 70042 PPH” refers to a pepper fruit containing maternal tissues of NUN 70042 PPH as deposited under Accession Number NCIMB ______. In one option, the fruit contains seed grown on NUN 70042 PPH. In another option, the fruit does not contain seed, that is the fruit is parthenocarpic. The skilled person is familiar with methods for inducing parthenocarpy. Those methods comprise chemically or genetically inducing parthenocarpy. Compounds suitable for chemically inducing parthenocarpy include auxins, gibberellins and cytokinins. Genetic parthenocarpy can a.o. be induced by CaARF8 mutants (Tiwari et al., BMC Plant Biology 201111:143 DOI: 10.1186/1471-2229-11-143) or as in WO 2013078319.

“Tissue culture” refers to a composition comprising isolated cells of the same or a different type or a collection of such cells organized into parts of a plant. Tissue culture of various tissues of pepper and regeneration of plants therefrom is well known and widely published (see, e.g., Sang-Gu et al. (1988), Plant Cell, Tissue and Organ Culture 12: 67-74; Kothari et al., (2010) Biotechnology Advances 28: 35-48. Similarly, the skilled person is well-aware how to prepare a “cell culture”.“

“UPOV descriptors” are the plant variety descriptors described for pepper in the “Guidelines for the Conduct of Tests for Distinctness, Uniformity and Stability, TG/76/8 (Geneva, 2006—updated 2015), as published by UPOV (International Union for the Protection of New Varieties and Plants, available on the world wide web at upov.int) and which can be downloaded from the world wide web at upov.int/under edocs/tgdocs/en/tg076.pdf and is herein incorporated by reference in its entirety.

“USDA descriptors” are the plant variety descriptors for pepper (Capsicum spp.) as published by the US Department of Agriculture, Agricultural Marketing Service, Plant Variety Protection Office, Beltsville, Md. 20705 (available on the world wide web at ams.usda.gov) and which can be downloaded from the world wide web atams.usda.gov/sites/default/files/media/56-Pepper.pdf.

“RHS” refers to the Royal Horticultural Society of England which publishes an official botanical color chart quantitatively identifying colors according to a defined numbering system. The chart may be purchased from Royal Horticulture Society Enterprise Ltd RHS Garden; Wisley, Woking; Surrey GU236QB, UK, e.g., the RHS colour chart: 2007 (The Royal Horticultural Society, charity No: 222879, PO Box 313 London SW1P2PE.

As used herein, the term “plant” includes the whole plant or any part or derivative thereof, preferably having the same genetic makeup as the plant from which it is obtained, such as a plant organ (e.g. harvested or non-harvested fruits), a plant cell, a plant protoplast, a plant cell tissue culture or a tissue culture from which a whole plant can be regenerated, a plant cell that is intact in a plant, a clone, a micropropagation, plant callus, a plant cell clump, a plant transplant, a vegetative propagation, a seedling, or parts of a plant (e.g. harvested tissues or organs), such as a fruit, a harvested fruit, a part of a fruit, a leaf, a part of a leaf, pollen, an ovule, an embryo, a petiole, a shoot or a part thereof, a stem or a part thereof, a root or a part thereof, a root tip, a cutting, a seed, a part of a seed, seedcoat or another maternal tissue which is part of a seed grown on a variety of the invention, hypocotyl, cotyledon, a scion, a graft, a stock, a rootstock, a pistil, an anther, and a flower or parts of any of these and the like. Also any developmental stage is included, such as seedlings, cuttings prior or after rooting, mature plants or leaves. Alternatively, a plant part may also include a plant seed which comprises one or two sets of chromosomes derived from the parent plant.

“Harvested plant material” refers herein to plant parts (e.g. fruits detached from the whole plant) which have been collected for further storage and/or further use.

“Harvested seeds” refers to seeds harvested from a line or variety, e.g. produced after self-fertilization or cross-fertilization and collected.

“REFERENCE VARIETY” refers to the variety Piton, a commercial variety from US Agriseeds, or to Forajido, a commercial variety from ENZA which have been planted in a trial together with NUN 70039 PPH or NUN 70041 PPH or NUN 70042 PPH. USDA descriptors of NUN 70039 PPH and NUN 70041 PPH were compared to the USDA descriptors of Piton. USDA descriptors of NUN 70042 PPH were compared to the USDA descriptors of Forajido.

“Rootstock” or “stock” refers to the plant selected for its roots, in particular for the resistance of the roots to diseases or stress (e.g. heat, cold, salinity etc.). Normally the quality of the fruit of the plant providing the rootstock is less important.

“Scion” refers to a part of the plant that is attached to the rootstock. This plant is selected for its stems, leaves, flowers, or fruits. The scion contains the desired genes to be duplicated in future production by the stock/scion plant and may produce the desired pepper fruit.

“Stock/scion” plant refers to a pepper plant comprising a rootstock from one plant grafted to a scion from another plant.

“Grafting” refers to attaching tissue from one plant to another plant so that the vascular tissues of the two tissues join together.

A plant having “all the physiological and morphological characteristics” of a referred-to-plant means a plant having the physiological and morphological characteristics of the referred-to-plant when grown under the same environmental conditions, preferably in the same experiment; the referred-to-plant can be a plant from which it was derived, e.g. the progenitor plant, the parent, the recurrent parent, the plant used for tissue- or cell culture, etc. A physiological or morphological characteristic can be a numerical characteristic or a non-numerical characteristic. In one aspect, a plant has “all but one, two or three of the physiological and morphological characteristics” of a referred-to-plant, or “all the physiological and morphological characteristics” of Table 1 and/or 2 or “all or all but one, two or three of the physiological and morphological characteristics” of Table 1 and/or 2

For NUN 70039 PPH the distinguishing characteristics are 1) average fruit flesh thickness; 2) average fruit calyx diameter; 3) average pedicel length; 4) presence of fruits with 2 locules; 5) average plant width; 6) average fruit placenta length; 7) average number of seeds per fruit; 8) average leaf petiole length; and 9) average number of calyx lobes.

For NUN 70041 PPH the distinguishing characteristics are 1) average fruit length; 2) average fruit flesh thickness at mid-point; 3) average number of seeds per fruit; 4) average pedicel length; 5) average flower diameter; 6) average leaf length; 7) average leaf petiole length; 8) typical leaf blistering; 9) average petiole diameter; and 10) absence of fruit with five or more locules.

For NUN 70042 PPH the distinguishing characteristics are 1) average fruit length; 2) average fruit width; 3) typical fruit apex shape; 4) average pedicel thickness; 5) average fruit seed cavity length; 6) average fruit seed cavity diameter; 7) typical leaf blistering; 8) typical style length; 9) average leaf length; and 10) average plant width.

In certain embodiments the plant of the invention has all the physiological and morphological characteristics, except for certain characteristics mentioned, e.g. the characteristic(s) derived from a converted or introduced gene or trait and/or except for the characteristics which differ, for example a Single Locus Conversion.

In one embodiment, the invention relates to a Single Locus Converted plant of NUN 70041 PPH.

Similarity between different plants is defined as the number of morphological and/or physiological characteristics (or the characteristics as listed in Table 1 and/or 2 that are the same between the two plants that are compared when grown under the same environmental conditions. Numerical characteristics are considered “the same” when the value for a numeric characteristic is evaluated at significance levels of 1%, 5% or 10% significance level, or at p≤0.05 using one way Analysis of variance (ANOVA), a standard methods known to the skilled person. Non-numerical or “type” characteristic are considered “the same” if identical or having the same value when scored for USDA and/or UPOV descriptors, if the plants are grown under the same conditions.

“Distinguishing characteristics” or “distinguishing morphological and/or physiological characteristics” refer herein the characteristics which are distinguishing between the new variety and other pepper varieties, such as the REFERENCE VARIETY for the new variety, when grown under the same environmental conditions. For NUN 70039 PPH, the following characteristics are especially relevant: 1) average fruit flesh thickness; 2) average fruit calyx diameter; 3) average pedicel length; 4) presence of fruits with 2 locules; 5) average plant width; 6) average fruit placenta length; 7) average number of seeds per fruit; 8) average leaf petiole length; and 9) average number of calyx lobes. For NUN 70041 PPH, the following characteristics are especially relevant: 1) average fruit length; 2) average fruit flesh thickness at mid-point; 3) average number of seeds per fruit; 4) average pedicel length; 5) average flower diameter; 6) average leaf length; 7) average leaf width; 8) typical leaf blistering; 9) average petiole diameter; and 10) absence of fruit with five or more locules, For NUN 70042 PPH, the following characteristics are especially relevant: 1) average fruit length; 2) average fruit width; 3) typical fruit apex shape; 4) average pedicel thickness; 5) average fruit seed cavity length; 6) average fruit seed cavity diameter; 7) typical leaf blistering; 8) typical style length; 9) average leaf length; and 10) average plant width. In one aspect, the distinguishing characteristics of each variety further include at least one, two, three or more (or all) of the characteristics listed in Table 1 and/or 2. All numerical distinguishing characteristics are statistically significantly different at p≤0.05 for the relevant REFERENCE VARIETY.

Thus, a pepper plant “comprising the distinguishing characteristics of “NUN 70039 PPH” refers herein to a pepper plant which does not differ significantly from NUN 70039 PPH in characteristics 1) to 9) above. In yet a further aspect the pepper plant of variety NUN 70039 PPH further does not differ in all or all but one, two, three, four, five or six characteristics listed in Table 1 and/or 2.

Further, a pepper plant “comprising the distinguishing characteristics of “NUN 70041 PPH” refers herein to a pepper plant which does not differ significantly from NUN 70041 PPH in characteristics 1) to 10) above. In yet a further aspect the pepper plant of variety NUN 70041 PPH further does not differ in all or all but one, two, three, four, five or six characteristics listed in Table 1 and/or 2.

Also, a pepper plant “comprising the distinguishing characteristics of “NUN 70042 PPH” refers herein to a pepper plant which does not differ significantly from NUN 70042 PPH in characteristics 1) to 10) above. In yet a further aspect the pepper plant of variety NUN 70042 PPH further does not differ in all or all but one, two, three, four, five or six characteristics listed in Table 1 and/or 2.

The physiological and/or morphological characteristics mentioned above are commonly evaluated at significance levels of 1%, 5% or 10% or evaluated at p≤0.05 using ANOVA, when measured under the same environmental conditions. For example, a progeny plant or a Single Locus Converted plant or a mutated plant of NUN 70039 PPH or NUN 70041 PPH or NUN 70042 PPH may have one or more (or all) of the essential physiological and/or morphological characteristics of NUN 70039 PPH or NUN 70041 PPH or NUN 70042 PPH listed in Table 1 and/or 2, as determined at the 5% significance level when grown under the same environmental conditions.

As used herein, the term “variety”, “cultivated pepper” or “cultivar” means a plant grouping within a single botanical taxon of the lowest known rank, which grouping, irrespective of whether the conditions for the grant of a breeder's right are fully met, can be defined by the expression of the characteristics resulting from a given genotype or combination of genotypes, distinguished from any other plant grouping by the expression of at least one of the said characteristics and considered as a unit with regard to its suitability for being propagated unchanged.

“Plant line” is for example a breeding line which can be used to develop one or more varieties. Progeny obtained by selfing a plant line has the same phenotype as its parents.

“Hybrid variety” or “F1 hybrid” refers to the seeds harvested from crossing two inbred (nearly homozygous) parental lines. For example, the female parent is pollinated with pollen of the male parent to produce hybrid (F1) seeds on the female parent.

“Regeneration” refers to the development of a plant from cell culture or tissue culture or vegetative propagation.

“Vegetative propagation”, “vegetative reproduction” or “clonal propagation” are used interchangeably herein and mean the method of taking part of a plant and allowing that plant part to form at least roots where plant part is, e.g., defined as or derived from (e.g. by cutting of) a fruit, a harvested fruit, a part of a fruit, a leaf, a part of a leaf, pollen, an ovule, a cell, a petiole, a shoot or a part thereof, a stem or a part thereof, a root or a part thereof, a root tip, a cutting, a seed, hypocotyl, cotyledon, a scion, a stock, a rootstock, a pistil, an anther, and a flower or a part thereof, etc. When a whole plant is regenerated by vegetative propagation, it is also referred to as a vegetative propagation.

“Selfing” refers to self-pollination of a plant, i.e., the transfer of pollen from the anther to the stigma of the same plant.

“Crossing” refers to the mating of two parent plants. The term encompasses “cross-pollination” and “selfing”.

In case of a hybrid, the trait may be introduced in the male or female parental line through backcrossing.

“Cross-pollination” refers to the fertilization by the union of two gametes from different plants.

“Planting” or “planted” refers to seeding (direct sowing) or transplanting seedlings (plantlets) into a field by machine or hand.

“Yield” means the total weight of all pepper fruits harvested per hectare of a particular line or variety. It is understood that “yield” expressed as weight of all pepper fruits harvested per hectare can be obtained by multiplying the number of plants per hectare times the “yield per plant”.

“Marketable yield” means the total weight of all marketable pepper fruits, especially fruit that is not cracked, damaged or diseased, harvested per hectare of a particular line or variety.

As used herein, the terms “resistance” and “tolerance” are used interchangeably to describe plants that show no symptoms or significantly reduced symptoms to a specified biotic pest, pathogen, abiotic influence or environmental condition. These terms are also used to describe plants showing some symptoms but that are still able to produce marketable product with an acceptable yield.

“Locus” (plural loci) refers to the specific location, place or site of a DNA sequence on a chromosome, where, for example, a gene or genetic marker is found. A locus may confer a specific trait.

“Allele” refers to one or more alternative forms of a gene locus. All of these loci relate to one trait. Sometimes, different alleles can result in different observable phenotypic traits, such as different pigmentation. However, many variations at the genetic level result in little or no observable variation. If a multicellular organism has two sets of chromosomes, i.e. diploid, these chromosomes are referred to as homologous chromosomes. Diploid organisms have one copy of each gene (and therefore one allele) on each chromosome. If both alleles are the same, they are homozygotes. If the alleles are different, they are heterozygotes.

“Genotype” refers to the genetic composition of a cell or organism.

“Phenotype” refers to the detectable characteristics of a plant, cell or organism, which characteristics are the manifestation of gene expression.

Haploid” refers to a cell or organism having one set of the two sets of chromosomes in a diploid.

“Diploid” refers to a cell or organism having two sets of chromosomes.

“Harvest maturity” is referred to as the stage at which a pepper fruit is ripe or ready for harvest or the optimal time to harvest the fruit. In one embodiment, harvest maturity is the stage which allows proper completion of the normal ripening.

“Fruit maturity” refers to the fruit developmental stage when the fruit has fully developed (reached its final size), begins to ripen and undergoes ripening, during which fruits can be divided into 1, 2, 3 or more maturity stages. Thereafter, fruits become overripe. In particular embodiments “maturity” is defined as the mature stage of fruit development and optimal time for harvest. In one embodiment a “mature” watermelon is defined as having reached the stage of maturity which will insure the proper completion of the normal ripening process. In particular embodiments, fruit should be harvested at a maturity stage i.e. substantially near maximum sweetness and flavor intensity.

“Flavor” (or flavour) refers to the sensory impression of a food or other substance, especially a pepper fruit or fruit part (fruit flesh) and is determined mainly by the chemical senses of taste and smell. Flavor is influenced by texture properties and by volatile and/or non-volatile chemical components (organic acids, lipids, carbohydrates, salts etc.).

Scoville Heat Unit (SHU) or Scoville Scale or Scoville Units is measurement of the pungency (spicy heat) of peppers as reported in Scoville heat units (SHU), a function of capsaicin concentration. The scale is named after its creator, American pharmacist Wilbur Scoville. The Scoville scale can be measured empirically, dependent on the capsaicin sensitivity of testers (i.e. by tasting the pepper or tasting diluted pepper samples until heat no longer can be detected by the tasters. Alternatively, high-performance liquid chromatography (HPLC) can used to determine SHU. In this procedure, fruits are dried and then ground, next, the chemicals responsible for heat are extracted, and the extract is injected into the HPLC for analysis.

The term “traditional breeding techniques” encompasses herein crossing, selfing, selection, double haploid production, embryo rescue, protoplast fusion, marker assisted selection, mutation breeding etc. as known to the breeder (i.e. methods other than genetic modification/transformation/transgenic methods), by which, for example, a genetically heritable trait can be transferred from one pepper line or variety to another. It optionally includes epigenetic modifications.

“Progeny” as used herein refers to a plant derived from a plant designated NUN 70039 PPH or a plant designated NUN 70041 PPH or a plant designated NUN 70042 PPH. A progeny may be derived by regeneration of cell culture or tissue culture or parts of a plant designated NUN 70039 PPH or a plant designated NUN 70041 PPH or a plant designated NUN 70042 PPH or selfing of a plant designated a plant designated NUN 70039 PPH or a plant designated NUN 70041 PPH or a plant designated NUN 70042 PPH or by producing seeds of a plant designated a plant designated NUN 70039 PPH or a plant designated NUN 70041 PPH or a plant designated NUN 70042 PPH. In further embodiments, progeny may also encompass plants derived from crossing of at least one plant designated a plant designated NUN 70039 PPH or a plant designated NUN 70041 PPH or a plant designated NUN 70042 PPH with another pepper plant of the same or another variety or (breeding) line, or wild pepper plants, backcrossing, inserting of a locus into a plant or mutation. A progeny is, e.g., a first generation progeny, i.e. the progeny is directly derived from, obtained from, obtainable from or derivable from the parent plant by, e.g., traditional breeding methods (selfing and/or crossing) or regeneration. However, the term “progeny” generally encompasses further generations such as second, third, fourth, fifth, sixth, seventh or more generations, i.e., generations of plants which are derived from, obtained from, obtainable from or derivable from the former generation by, e.g., traditional breeding methods, regeneration or genetic transformation techniques. For example, a second generation progeny can be produced from a first generation progeny by any of the methods mentioned above.

The terms “gene converted” or “conversion plant” in this context refer to pepper plants which are developed by backcrossing wherein essentially all of the desired morphological and physiological characteristics of parent are recovered in addition to the one or more genes transferred into the parent via the backcrossing technique or via genetic engineering. Likewise a “Single Locus Converted (Conversion) Plant” refers to plants which are developed by plant breeding techniques comprising or consisting of backcrossing, wherein essentially all of the desired morphological and physiological characteristics of a pepper variety are recovered in addition to the characteristics of the single locus having been transferred into the variety via the backcrossing technique and/or by genetic transformation. In case of a hybrid, the gene may be introduced in the male or female parental line.

“Transgene” or “chimeric gene” refers to a genetic locus comprising a DNA sequence which has been introduced into the genome of a pepper plant by transformation. A plant comprising a transgene stably integrated into its genome is referred to as “transgenic plant”.

“Marker” refers to a readily detectable phenotype, preferably inherited in codominant fashion (both alleles at a locus in a diploid heterozygote are readily detectable), with no environmental variance component, i.e., a heritability of 1.

“Average” refers herein to the arithmetic mean.

“Substantially equivalent” refers to a characteristic that, when compared, does not show a statistically significant difference (e.g., p=0.05) from the mean.

The term “mean” refers to the arithmetic mean of several measurements. The skilled person understands that the appearance of a plant depends to some extent on the growing conditions of said plant. Thus, the skilled person will know typical growing conditions for peppers described herein. The mean, if not indicated otherwise within this application, refers to the arithmetic mean of measurements on at least 10 different, randomly selected plants of a variety or line.

### DETAILED DESCRIPTION

The present invention relates to a Capsicum annuum variety, referred to as NUN 70039 PPH, which—when compared to its REFERENCE VARIETY Piton—has a: 1) higher average fruit flesh thickness; 2) lower average fruit calyx diameter; 3) higher average pedicel length; 4) presence of fruits with 2 locules; 5) higher average plant width; 6) lower average fruit placenta length; 7) lower average number of seeds per fruit; 8) higher average leaf petiole length; and 9) higher average number of calyx lobes. Also encompassed by the present invention are progeny plants having all but 1, 2, or 3 of the morphological and/physiological characteristics of NUN 70039 PPH and methods of producing plants in accordance with the present invention.

The present invention also relates to a Capsicum annuum variety, referred to as NUN 70041 PPH, which—when compared to its check variety REFERENCE VARIETY Piton—has a 1) lower average fruit length; 2) higher average fruit flesh thickness at mid-point; 3) average number of seeds per fruit; 4) higher average pedicel length; 5) higher average flower diameter; 6) lower average leaf length; 7) higher average leaf petiole length; 8) typically absent leaf blistering; 9) lower average petiole diameter; and 10) absence of fruit with five or more locules. Also encompassed by the present invention are progeny plants having all but 1, 2, or 3 of the morphological and/physiological characteristics of NUN 70041 PPH and methods of producing plants in accordance with the present invention.

The present invention further relates to a Capsicum annuum variety, referred to as NUN 70042 PPH, which—when compared to check variety its REFERENCE VARIETY Forajido—has a 1) average fruit length; 2) average fruit width; 3) typical fruit apex shape; 4) average pedicel thickness; 5) average fruit seed cavity length; 6) average fruit seed cavity diameter; 7) typically very weak leaf blistering; 8) typical style length; 9) lower average leaf length; and 10) lower average plant width. Also encompassed by the present invention are progeny plants having all but 1, 2, or 3 of the morphological and/physiological characteristics of NUN 70042 PPH and methods of producing plants in accordance with the present invention.

Thus, in one aspect, the invention provides a seed of the pepper variety designated NUN 70039 PPH wherein a representative sample of seeds of said variety was deposited under the Budapest Treaty, with Accession number NCIMB ______.

Thus, in one more aspect, the invention provides a seed of the pepper variety designated NUN 70041 PPH wherein a representative sample of seeds of said variety was deposited under the Budapest Treaty, with Accession number NCIMB ______.

Thus, in another aspect, the invention provides a seed of the pepper variety designated NUN 70042 PPH wherein a representative sample of seeds of said variety was deposited under the Budapest Treaty, with Accession number NCIMB ______.

In another aspect, the invention provides for a pepper plant of variety NUN 70039 PPH, a representative sample of seed from said variety has been deposited under the Budapest Treaty, with Accession number NCIMB ______.

In one more aspect, the invention provides for a pepper plant of variety NUN 70041 PPH, a representative sample of seed from said variety has been deposited under the Budapest Treaty, with Accession number NCIMB ______.

In also an aspect, the invention provides for a pepper plant of variety NUN 70042 PPH, a representative sample of seed from said variety has been deposited under the Budapest Treaty, with Accession number NCIMB ______.

A seed of NUN 70039 PPH is obtainable by crossing the male parent of NUN 70039 PPH with the female parent of NUN 70039 PPH and harvesting the seeds produced on the female parent. The resultant NUN 70039 PPH seeds can be grown to produce NUN 70039 PPH plants. In one embodiment a seed or a plurality of seeds of NUN 70039 PPH is packaged into a container of any size or type (e.g., bags, cartons, cans, etc.). The seed may be disinfected, primed and/or treated with various compounds, such as seed coatings or crop protection compounds.

Also, a seed of NUN 70041 PPH is obtainable by crossing the male parent of NUN 70041 PPH with the female parent of NUN 70041 PPH and harvesting the seeds produced on the female parent. The resultant NUN 70041 PPH seeds can be grown to produce NUN 70041 PPH plants. In one embodiment a seed or a plurality of seeds of NUN 70041 PPH is packaged into a container of any size or type (e.g., bags, cartons, cans, etc.). The seed may be disinfected, primed and/or treated with various compounds, such as seed coatings or crop protection compounds.

Further, a seed of NUN 70042 PPH is obtainable by crossing the male parent of NUN 70042 PPH with the female parent of NUN 70042 PPH and harvesting the seeds produced on the female parent. The resultant NUN 70042 PPH seeds can be grown to produce NUN 70042 PPH plants. In one embodiment a seed or a plurality of seeds of NUN 70042 PPH is packaged into a container of any size or type (e.g., bags, cartons, cans, etc.). The seed may be disinfected, primed and/or treated with various compounds, such as seed coatings or crop protection compounds.

Also provided is a plant of pepper variety NUN 70039 PPH, or a fruit or other plant part thereof, produced from a seed, wherein a representative sample of said seeds has been deposited under the Budapest Treaty, with Accession Number NCIMB ______. Also included is a cell culture or tissue culture produced from such a plant.

Also provided is a plant of pepper variety NUN 70041 PPH, or a fruit or other plant part thereof, produced from a seed, wherein a representative sample of said seeds has been deposited under the Budapest Treaty, with Accession Number NCIMB ______. Also included is a cell culture or tissue culture produced from such a plant.

Also provided is a plant of pepper variety NUN 70042 PPH, or a fruit or other plant part thereof, produced from a seed, wherein a representative sample of said seeds has been deposited under the Budapest Treaty, with Accession Number NCIMB ______. Also included is a cell culture or tissue culture produced from such a plant.

In one embodiment the invention provides a pepper plant regenerated from the tissue or cell culture of NUN 70039 PPH or NUN 70041 PPH or NUN 70042 PPH, wherein the plant has all or all but one, two or three of of the physiological and morphological characteristics of the new variety as listed in Table 1 and/or 2. In another embodiment, the invention provides a pepper plant regenerated from the tissue or cell culture of NUN 70039 PPH or NUN 70041 PPH or NUN 70042 PPH PPH, wherein the plant has all or all but one, two or three of the physiological and morphological characteristics of the new variety. In these cases, similarity of a characteristic is determined by measuring a representative number of plants grown under the same conditions, where type characteristics are the same and numerical characteristics are substantially equivalent (determined at the 5% significance level or evaluated at p≤0.05 using ANOVA).

A plant of NUN 70039 PPH or NUN 70041 PPH or NUN 70042 PPH can be produced by seeding directly in the soil (e.g., field) or by germinating the seeds in controlled environment conditions (e.g., greenhouses) and then transplanting the seedlings into the field. For example, the seed can be sown into prepared seed beds where they will remain for the entire production of the crop. Alternatively, the pepper seed may be planted through a black plastic mulch. The dark plastic will absorb heat from the sun, warming the soil early. It will also help to conserve moisture during the growing season, controls weeds and makes harvesting easier and cleaner. Pepper can also be grown entirely in greenhouses. See for example J. Burt, Farmnote 64/99 for cultivation, harvesting, handling and postharvest methods commonly used.

In other aspects, the invention provides for a fruit or a part thereof of pepper variety NUN 70039 PPH or NUN 70041 PPH or NUN 70042 PPH, or a plant part, such as pollen, flowers, shoots or cuttings of variety NUN 70039 PPH or NUN 70041 PPH or NUN 70042 PPH or parts thereof.

In one embodiment any plant of NUN 70039 PPH comprises at least 3, 4, 5 or more, e.g. 6, 7, 8, 9 or all of the following morphological and/or physiological characteristics (i.e. distinguishing as indicated on the USDA Objective description of variety—Pepper listed in Table 1 and/or 2, when grown under the same environmental conditions): 1) average fruit flesh thickness; 2) average fruit calyx diameter; 3) average pedicel length; 4) presence of fruits with 2 locules; 5) average plant width; 6) average fruit placenta length; 7) average number of seeds per fruit; 8) average leaf petiole length; and 9) average number of calyx lobes

In one embodiment any plant of NUN 70041 PPH comprises at least 3, 4, 5 or more, e.g. 6, 7, 8, 9 or all of the following morphological and/or physiological characteristics (i.e. distinguishing as indicated on the USDA Objective description of variety—Pepper listed in Table 1 and/or 2, when grown under the same environmental conditions): 1) average fruit length; 2) average fruit flesh thickness at mid-point; 3) average number of seeds per fruit; 4) average pedicel length; 5) average flower diameter; 6) average leaf length; 7) average leaf petiole length; 8) typical leaf blistering; 9) average petiole diameter; and 10) absence of fruit with five or more locules.

In one embodiment any plant of NUN 70042 PPH comprises at least 3, 4, 5 or more, e.g. 6, 7, 8, 9 or all of the following morphological and/or physiological characteristics (i.e. distinguishing as indicated on the USDA Objective description of variety—Pepper listed in Table 1 and/or 2, when grown under the same environmental conditions): 1) average fruit length; 2) average fruit width; 3) typical fruit apex shape; 4) average pedicel thickness; 5) average fruit seed cavity length; 6) average fruit seed cavity diameter; 7) typical leaf blistering; 8) typical style length; 9) average leaf length; and 10) average plant width.

In still another aspect the invention provides a method of producing a pepper plant, comprising crossing a plant of pepper variety NUN 70039 PPH or NUN 70041 PPH or NUN 70042 PPH with a second pepper plant one or more times, and selecting progeny from said crossing. In one embodiment of the invention, the first step in “crossing” comprises planting seeds of a first and a second parent pepper plant, often in proximity so that pollination will occur for example, mediated by insect vectors. Alternatively, pollen can be transferred manually. Where the plant is self-pollinated, pollination may occur without the need for direct human intervention other than plant cultivation.

In yet another aspect the invention provides a method of producing a pepper plant, comprising selfing a plant of pepper variety NUN 70039 PPH or NUN 70041 PPH or NUN 70042 PPH one or more times, and selecting progeny from said selfing.

In other aspects, the invention provides for a progeny of variety NUN 70039 PPH or NUN 70041 PPH or NUN 70042 PPH such as progeny obtained by further breeding that variety of the invention. Further breeding any one of the pepper varieties of the invention includes selfing that variety of the invention one or more times and/or cross-pollinating that variety of the invention with another pepper plant or variety one or more times. In particular, the invention provides for progeny that retain all the essential morphological and physiological characteristics of that variety of the invention or that retain one or more of the distinguishing characteristics of a variety of the invention described further above and when grown under the same environmental conditions. In another aspect, the invention provides for a vegetative reproduction of the variety and a plant having all but 1, 2, or 3 of the physiological and morphological characteristics of NUN 70039 PPH or NUN 70041 PPH or NUN 70042 PPH (e.g. as listed in Table 1 and/or 2).

The morphological and/or physiological differences between a plant of one of the varieties of the invention, i.e. NUN 70039 PPH or NUN 70041 PPH or NUN 70042 PPH or progeny of any of these varieties, or plants having all but 1, 2, or 3 of the physiological and morphological characteristics of NUN 70039 PPH or NUN 70041 PPH or NUN 70042 PPH (as listed in Table 1 and/or 2); and other known varieties can easily be established by growing any one of the varieties of the invention, i.e. NUN 70039 PPH or NUN 70041 PPH or NUN 70042 PPH, next to the other variety or varieties (in the same field, under the same environmental conditions), preferably in several locations suitable for pepper cultivation, and measuring morphological and/or physiological characteristics of a number of plants (e.g., to calculate an average value and to determine the variation range/uniformity within the variety). For example, trials can be carried out in Acampo Calif., USA (N 38 degrees 07′261″/W 121 degrees 18′ 807″, USA, whereby, maturity, days from seeding to harvest, plant habit, plant attitude, leaf shape, leaf color, blistering, numbers of flowers per leaf axil, number of calyx lobes, number of petals, fruit group, immature fruit color, mature fruit color, pungency, flavor, fruit glossiness, fruit size, fruit shape, average number of fruits per plant, seed size, seed weight, anthocyanin level, disease resistance, insect resistance, can be measured and directly compared for species of Capsicum.

The morphological and physiological characteristics (and the distinguishing characteristics) of NUN 70039 PPH or NUN 70041 PPH or NUN 70042 PPH are provided in the Examples, in Table 1 and/or 2. Encompassed herein is also a plant derivable from NUN 70039 PPH or NUN 70041 PPH or NUN 70042 PPH (e.g. by selfings and/or crossing and/or backcrossing with NUN 70039 PPH or NUN 70041 PPH or NUN 70042 PPH and/or progeny of said varieties) comprising all or all but one, two or three of the physiological and morphological characteristics of NUN 70039 PPH or NUN 70041 PPH or NUN 70042 PPH listed in Table 1 and/or 2 are similar (as defined in this application) and/or comprising one or more (or all; or all except one, two or three) of the distinguishing characteristics as determined at the 5% significance level when grown under the same environmental conditions.

Also at-harvest and/or post-harvest characteristics of fruits can be compared, such as cold storage holding quality, post-harvest flesh firmness, heat (for example in Scoville units) and Brix can be measured using known methods. Flesh firmness can for example be measured using a penetrometer, e.g. by inserting a probe into the fruit flesh and determining the insertion force, or by other methods.

The morphological and/or physiological characteristics may vary somewhat with variation in the environment (such as temperature, light intensity, day length, humidity, soil, fertilizer use), which is why a comparison under the same environmental conditions is preferred. Colors can best be measured against The Munsell Book of Color (Munsell Color Macbeth Division of Kollmorgan Instruments Corporation) or using the Royal Horticultural Society Chart (World Wide Web at rhs.org.uk/Plants/RHS-Publications/RHS-colour-charts).

In a preferred embodiment, the invention provides for a pepper fruit of variety NUN 70039 PPH or NUN 70041 PPH or NUN 70042 PPH, or a part of a fruit of said varieties. In another embodiment, the invention provides for a container comprising or consisting of a plurality of harvested pepper fruits or parts of fruits of NUN 70039 PPH or NUN 70041 PPH or NUN 70042 PPH, or fruits of progeny of said varieties, or fruits of a derived variety.

In yet a further embodiment, the invention provides for a method of producing a new pepper plant. The method comprises crossing a plant of the invention i.e. NUN 70039 PPH or NUN 70041 PPH or NUN 70042 PPH, or a plant comprising all but 1, 2, or 3 of the morphological and physiological characteristics of NUN 70039 PPH or NUN 70041 PPH or NUN 70042 PPH (as listed in Table 1 and/or 2), or a progeny plant thereof, either as male or as female parent, with a second pepper plant (or a wild relative of pepper) one or more times, and/or selfing a pepper plant according to the invention i.e. NUN 70039 PPH or NUN 70041 PPH or NUN 70042 PPH, or a progeny plant thereof, one or more times, and selecting progeny from said crossing and/or selfing. The second pepper plant may for example be a line or variety of the species Capsicum annuum, C. frutecens, C. baccatum, C. chinense, or other Capsicum species.

Progeny are either the generation (seeds) produced from the first cross (F1) or selfing (S1), or any further generation produced by crossing and/or selfing (F2, F3, etc.) and/or backcrossing (BC1, BC2, etc.) one or more selected plants of the F1 and/or S1 and/or BC1 generation (or plants of any further generation, e.g. the F2) with another pepper plant (and/or with a wild relative of pepper). Progeny may have all the physiological and morphological characteristics of pepper variety NUN 70039 PPH or NUN 70041 PPH or NUN 70042 PPH when grown under the same environmental conditions and/or progeny may have (be selected for having) one or more of the distinguishing characteristics of pepper of the invention. Using common breeding methods such as backcrossing or recurrent selection, one or more specific characteristics may be introduced into NUN 70039 PPH or NUN 70041 PPH or NUN 70042 PPH, to provide or a plant comprising all but 1, 2, or 3 or more of the morphological and physiological characteristics of NUN 70039 PPH or NUN 70041 PPH or NUN 70042 PPH (as listed in Table 1 and/or 2).

The invention provides for methods of producing plants which retain all the morphological and physiological characteristics of a plant of the invention i.e. NUN 70039 PPH or NUN 70041 PPH or NUN 70042 PPH. The invention provides also for methods of producing a plant comprising all but 1, 2, or 3 or more of the morphological and physiological characteristics of NUN 70039 PPH or NUN 70041 PPH or NUN 70042 PPH (e.g. as listed in Table 1 and/or 2), but which are still genetically closely related to NUN 70039 PPH or NUN 70041 PPH or NUN 70042 PPH. The relatedness can, for example, be determined by fingerprinting techniques (e.g., making use of isozyme markers and/or molecular markers such as SNP markers, AFLP markers, microsatellites, minisatellites, RAPD markers, RFLP markers and others). A plant is “closely related” to NUN 70039 PPH or NUN 70041 PPH or NUN 70042 PPH if its DNA fingerprint is at least 80%, 90%, 95% or 98% identical to the fingerprint of NUN 70039 PPH or NUN 70041 PPH or NUN 70042 PPH. In a preferred embodiment AFLP markers are used for DNA fingerprinting (Vos et al. 1995, Nucleic Acid Research 23: 4407-4414). A closely related plant may have a Jaccard's Similarity index of at least about 0.8, preferably at least about 0.9, 0.95, 0.98 or more (Ince et al., (2010) Biochem. Genet. 48:83-95). The invention also provides plants and varieties obtained or selected by applying these methods on NUN 70039 PPH or NUN 70041 PPH or NUN 70042 PPH. Plants may be produced by crossing and/or selfing, or alternatively, a plant may simply be identified and selected amongst NUN 70039 PPH or NUN 70041 PPH or NUN 70042 PPH plants, or progeny of said varieties, e.g. by identifying a variant within NUN 70039 PPH or NUN 70041 PPH or NUN 70042 PPH or progeny of said varieties (e.g. produced by selfing) which variant differs from NUN 70039 PPH or NUN 70041 PPH or NUN 70042 PPH in one, two or three of the morphological and/or physiological characteristics (e.g. in one, two or three distinguishing characteristics), e.g. those listed in Table 1 and/or 2 or others. In one embodiment the invention provides a pepper plant having a Jaccard's Similarity index with NUN 70039 PPH or NUN 70041 PPH or NUN 70042 PPH of at least 0.8, e.g. at least 0.85, 0.9, 0.95, 0.98 or even at least 0.99.

The present invention also provides a pepper seed and a plant produced by a process that comprises crossing a first parent pepper plant with a second parent pepper plant, wherein at least one of the first or second parent pepper plants is a plant provided herein, such as from variety NUN 70039 PPH or NUN 70041 PPH or NUN 70042 PPH. In another embodiment of the invention, pepper seed and plants produced by the process are first filial generation (F1) pepper seed and plants produced by crossing a plant in accordance with the invention with another, distinct plant.

The present invention further contemplates plant parts of such an F1 pepper plant, and methods of use thereof. Therefore, certain exemplary embodiments of the invention provide an F1 pepper plant and seed thereof.

WO2013182646 which is incorporated by reference, relates to a non-destructive method for analyzing maternal DNA of a seed. In this method the DNA is dislodged from the seed coat surface and can be used to collect information on the genome of the maternal parent of the seed. This method for analyzing maternal DNA of a seed, comprises the steps of contacting a seed with a fluid to dislodge DNA from the seed coat surface, and analyzing the DNA thus dislodged from the seed coat surface using methods known in the art. The skilled person is thus able to determine whether a seed has grown on a plant of a plant of the invention i.e. NUN 70039 PPH or NUN 70041 PPH or NUN 70042 PPH (i.e. is progeny of NUN 70039 PPH or NUN 70041 PPH or NUN 70042 PPH), because the seed coat is genetically identical to NUN 70039 PPH or NUN 70041 PPH or NUN 70042 PPH. In one embodiment, the present invention relates to a seed coat comprising maternal tissue of NUN 70039 PPH or NUN 70041 PPH or NUN 70042 PPH. In another embodiment the invention relates to a pepper seed comprising a seed coat that comprises maternal tissue from NUN 70039 PPH or NUN 70041 PPH or NUN 70042 PPH.

By crossing and/or selfing also (one or more) single traits may be introduced into the variety of the invention i.e. NUN 70039 PPH or NUN 70041 PPH or NUN 70042 PPH (e.g., using backcrossing breeding schemes), while retaining the remaining morphological and physiological characteristics of NUN 70039 PPH or NUN 70041 PPH or NUN 70042 PPH and/or while retaining one or more distinguishing characteristics. A single trait converted plant may thereby be produced. For example, disease resistance genes may be introduced, genes responsible for one or more quality traits, yield, etc. Both single genes (dominant or recessive) and one or more QTLs (quantitative trait loci) may be transferred into NUN 70039 PPH or NUN 70041 PPH or NUN 70042 PPH by breeding with NUN 70039 PPH or NUN 70041 PPH or NUN 70042 PPH.

Alternatively, a single trait converted plant or single locus converted plant of the invention i.e. NUN 70039 PPH or NUN 70041 PPH or NUN 70042 PPH may be produced by the following steps
a. obtaining a cell or tissue culture of cells of NUN 70039 PPH or NUN 70041 PPH or NUN 70042 PPH;
b. genetically transforming or mutating said cells;
c. growing the cells into a plant; and
d. optionally selecting a plant that contains the desired single locus conversion
   The skilled person is familiar with various techniques for genetically transforming a single locus in a plant cell, or mutating said cells.

Any pest or disease resistance genes may be introduced into a plant according to the invention, i.e. NUN 70039 PPH or NUN 70041 PPH or NUN 70042 PPH, progeny of said varieties or into a plant comprising all but 1, 2, or 3 or more of the morphological and physiological characteristics of NUN 70039 PPH or NUN 70041 PPH or NUN 70042 PPH (e.g. as listed in Table 1 and/or 2). Resistance to one or more of the following diseases is preferably introduced into plants of the invention: Cucumber Mosaic Virus, Curly Top Virus, Pepper Mottle Virus, Potato Y Virus, Tobacco Etch Virus, Tobacco Mosaic Virus, Anthracnose (Gloeosporium piperatum), Bacterial Spot (Xanthomonas vesicatoria), Cercospora Leaf Spot (Cercospora capsici), Nematode (Meloidogyne incognita acrita), Phytophthora Root Rot (Phytophthora capsici), Ripe Rot (Vermicularia capsici), Southern Blight (Sclerotium rolfsii) and/or Verticillium Wilt (Verticillium dahliae). Other resistance genes, against pathogenic viruses, fungi, bacteria, nematodes, insects or other pests may also be introduced. In an embodiment, the resistance is TSWV resistance.

Thus, invention also provides a method for developing a pepper plant in a pepper breeding program, using a pepper plant of the invention, or its parts as a source of plant breeding material. Suitable plant breeding techniques are recurrent selection, backcrossing, pedigree breeding, mass selection, mutation breeding and/or genetic marker enhanced selection. For example, in one aspect, the method comprises crossing into a plant according to the invention, i.e. NUN 70039 PPH or NUN 70041 PPH or NUN 70042 PPH, or progeny of said varieties, or a plant comprising all but 1, 2, or 3 or more of the morphological and physiological characteristics of 70039 PPH or NUN 70041 PPH or NUN 70042 PPH (e.g. as listed in Table 1 and/or 2), with a different pepper plant, and wherein one or more offspring of the crossing are subject to one or more plant breeding techniques selected from the group consisting of recurrent selection, backcrossing, pedigree breeding, mass selection, mutation breeding and genetic marker enhanced selection (see e.g. see e.g. Thabuis et al., (2004) Theor Appl Genet 109:342-351). For breeding methods in general see Principles of Plant Genetics and Breeding, 2007, George Acquaah, Blackwell Publishing, ISBN-13: 978-1-4051-3646-4.

The invention also provides a pepper plant comprising at least a first set of the chromosomes of pepper variety NUN 70039 PPH, a sample of seed of said variety having been deposited under Accession Number NCIMB ______; optionally further comprising a single locus conversion or a mutation, wherein said plant has essentially all of the morphological and physiological characteristics of the plant comprising at least a first set of the chromosomes of pepper NUN 70039 PPH. In another embodiment, this single locus conversion confers a trait selected from the group consisting of male sterility, herbicide tolerance, insect resistance, pest resistance, disease resistance, environmental stress tolerance, modified carbohydrate metabolism and modified protein metabolism.

The invention also provides a pepper plant comprising at least a first set of the chromosomes of pepper variety NUN 70041 PPH, a sample of seed of said variety having been deposited under Accession Number NCIMB ______; optionally further comprising a single locus conversion or a mutation, wherein said plant has essentially all of the morphological and physiological characteristics of the plant comprising at least a first set of the chromosomes of pepper NUN 70041 PPH. In another embodiment, this single locus conversion confers a trait selected from the group consisting of male sterility, herbicide tolerance, insect resistance, pest resistance, disease resistance, environmental stress tolerance, modified carbohydrate metabolism and modified protein metabolism.

The invention also provides a pepper plant comprising at least a first set of the chromosomes of pepper variety NUN 70042 PPH, a sample of seed of said variety having been deposited under Accession Number NCIMB ______; optionally further comprising a single locus conversion or a mutation, wherein said plant has essentially all of the morphological and physiological characteristics of the plant comprising at least a first set of the chromosomes of pepper NUN 70042 PPH. In another embodiment, this single locus conversion confers a trait selected from the group consisting of male sterility, herbicide tolerance, insect resistance, pest resistance, disease resistance, environmental stress tolerance, modified carbohydrate metabolism and modified protein metabolism.

In one embodiment, a plant according to the invention, i.e. NUN 70039 PPH or NUN 70041 PPH or NUN 70042 PPH, may also be mutated (by e.g. irradiation, chemical mutagenesis, heat treatment, etc.) and mutated seeds or plants may be selected in order to change one or more characteristics of NUN 70039 PPH or NUN 70041 PPH or NUN 70042 PPH. Methods such as TILLING may be applied to pepper populations in order to identify mutants. Similarly, NUN 70041 PPH may be transformed and regenerated, whereby one or more chimeric genes are introduced into the variety or into a plant comprising all but 1, 2, 3, or more of the morphological and physiological characteristics (e.g. as listed in Table 1 and/or 2). Transformation can be carried out using standard methods, such as Agrobacterium tumefaciens mediated transformation or biolistics, followed by selection of the transformed cells and regeneration into plants. A desired trait (e.g. genes conferring pest or disease resistance, herbicide, fungicide or insecticide tolerance, etc.) can be introduced into NUN 70039 PPH or NUN 70041 PPH or NUN 70042 PPH, or progeny of said varieties, by transforming NUN 70039 PPH or NUN 70041 PPH or NUN 70042 PPH or progeny of said varieties with a transgene that confers the desired trait, wherein the transformed plant retains all the phenotypic and/or morphological and/or physiological characteristics of NUN 70039 PPH or NUN 70041 PPH or NUN 70042 PPH or the progeny of said varieties and contains the desired trait.

The invention also provides a plant or a cell of a pepper plant comprising a desired trait produced by mutating a plant of variety NUN 70039 PPH or NUN 70041 PPH or NUN 70042 PPH or a cell thereof and selecting a plant the desired trait, wherein the mutated plant retains all or all but one of the phenotypic and morphological characteristics of said variety, optionally as described for each variety in in Table 1 and/or 2, and contains the desired trait and wherein a representative sample of seed of variety NUN 70039 PPH has been deposited under Accession Number NCIMB ______, seed of variety NUN 70041 PPH has been deposited under Accession Number NCIMB ______ and seed of variety NUN 70042 PPH has been deposited under Accession Number NCIMB ______. In a further embodiment, the desired trait is selected from the group consisting of fruit quality, male sterility, herbicide tolerance, insect resistance, pest resistance, disease resistance, virus resistance, TSWV resistance, Ve427^{RB} resistance, environmental stress tolerance, modified carbohydrate metabolism, modified protein metabolism and ripening or the mutation occurs in any of the following genes or genetic elements: genetic element which genetic element comprises a Ve427^{RB}-resistance conferring QTL.

The invention also provides a plant having one, two or three physiological and/or morphological characteristics which are different from those of NUN 70039 PPH and which otherwise has all the physiological and morphological characteristics of NUN 70039 PPH, wherein a representative sample of seed of variety NUN 70039 PPH has been deposited under Accession Number NCIMB ______. In particular variants which differ from NUN 70039 PPH in none, one, two or three of the characteristics mentioned in Table 1 and/or 2 are encompassed.

The invention also provides a plant having one, two or three physiological and/or morphological characteristics which are different from those of NUN 70041 PPH and which otherwise has all the physiological and morphological characteristics of NUN 70041 PPH, wherein a representative sample of seed of variety NUN 70041 PPH has been deposited under Accession Number NCIMB ______. In particular variants which differ from NUN 70041 PPH in none, one, two or three of the characteristics mentioned in Table 1 and/or 2 are encompassed.

The invention also provides a plant having one, two or three physiological and/or morphological characteristics which are different from those of NUN 70042 PPH and which otherwise has all the physiological and morphological characteristics of NUN 70042 PPH, wherein a representative sample of seed of variety NUN 70042 PPH has been deposited under Accession Number NCIMB ______. In particular variants which differ from NUN 70042 PPH in none, one, two or three of the characteristics mentioned in Table 1 and/or 2 are encompassed.

In one aspect, the the plant having one, two or three physiological and/or morphological characteristics which are different from those of NUN 70039 PPH and which otherwise has all the physiological and morphological characteristics of NUN 70039 PPH differs from NUN 70039 PPH in one, two or three of the distinguishing morphological and/or physiological characteristics selected from 1) average fruit flesh thickness; 2) average fruit calyx diameter; 3) average pedicel length; 4) presence of fruits with 2 locules; 5) average plant width; 6) average fruit placenta length; 7) average number of seeds per fruit; 8) average leaf petiole length; and 9) average number of calyx lobes.

In one aspect, the the plant having one, two or three physiological and/or morphological characteristics which are different from those of NUN 70041 PPH and which otherwise has all the physiological and morphological characteristics of NUN 70041 PPH differs from NUN 70041 PPH in one, two or three of the distinguishing morphological and/or physiological characteristics selected from 1) average fruit length; 2) average fruit flesh thickness at mid-point; 3) average number of seeds per fruit; 4) average pedicel length; 5) average flower diameter; 6) average leaf length; 7) average leaf petiole length; 8) typical leaf blistering; 9) average petiole diameter; and 10) absence of fruit with five or more locules.

In one aspect, the the plant having one, two or three physiological and/or morphological characteristics which are different from those of NUN 70042 PPH and which otherwise has all the physiological and morphological characteristics of NUN 70042 PPH differs from NUN 70042 PPH in one, two or three of the distinguishing morphological and/or physiological characteristics selected from 1) average fruit length; 2) average fruit width; 3) typical fruit apex shape; 4) average pedicel thickness; 5) average fruit seed cavity length; 6) average fruit seed cavity diameter; 7) typical leaf blistering; 8) typical style length; 9) average leaf length; and 10) average plant width.

In another embodiment the plant having one, two or three physiological and/or morphological characteristics which are different from those of NUN 70039 PPH and which otherwise has all the physiological and morphological characteristics of NUN 70039 PPH may differ from NUN 70039 PPH in one, two or three morphological or physiological characteristic other than the “distinguishing morphological and/or physiological characteristics” (or essential physiological and/or morphological characteristics) of NUN 70039 PPH selected from: 1) average fruit flesh thickness; 2) average fruit calyx diameter; 3) average pedicel length; 4) presence of fruits with 2 locules; 5) average plant width; 6) average fruit placenta length; 7) average number of seeds per fruit; 8) average leaf petiole length; and 9) average number of calyx lobes.

In another embodiment the plant having one, two or three physiological and/or morphological characteristics which are different from those of NUN 70041 PPH and which otherwise has all the physiological and morphological characteristics of NUN 70041 PPH may differ from NUN 70041 PPH in one, two or three morphological or physiological characteristic other than the “distinguishing morphological and/or physiological characteristics” (or essential physiological and/or morphological characteristics) of NUN 70041 PPH selected from: 1) average fruit length; 2) average fruit flesh thickness at mid-point; 3) average number of seeds per fruit; 4) average pedicel length; 5) average flower diameter; 6) average leaf length; 7) average leaf petiole length; 8) typical leaf blistering; 9) average petiole diameter; and 10) absence of fruit with five or more locules.

In another embodiment the plant having one, two or three physiological and/or morphological characteristics which are different from those of NUN 70042 PPH and which otherwise has all the physiological and morphological characteristics of NUN 70042 PPH may differ from NUN 70042 PPH in one, two or three morphological or physiological characteristic other than the “distinguishing morphological and/or physiological characteristics” (or essential physiological and/or morphological characteristics) of NUN 70042 PPH selected from: 1) average fruit length; 2) average fruit width; 3) typical fruit apex shape; 4) average pedicel thickness; 5) average fruit seed cavity length; 6) average fruit seed cavity diameter; 7) typical leaf blistering; 8) typical style length; 9) average leaf length; and 10) average plant width.

Peppers according to the invention, such as the varieties NUN 70039 PPH or NUN 70041 PPH or NUN 70042 PPH, or its progeny, or a plant having all physiological and/or morphological characteristics but one, two or three which are different from those of NUN 70039 PPH or NUN 70041 PPH or NUN 70042 PPH, can also be reproduced using vegetative reproduction methods. Therefore, the invention provides for a method of producing a plant, or a part thereof, of variety NUN 70039 PPH or NUN 70041 PPH or NUN 70042 PPH, comprising vegetative propagation of variety NUN 70039 PPH or NUN 70041 PPH or NUN 70042 PPH. Vegetative propagation comprises regenerating a whole plant from a plant part of variety NUN 70039 PPH or NUN 70041 PPH or NUN 70042 PPH (or from a progeny of any of NUN 70039 PPH or NUN 70041 PPH or NUN 70042 PPH or from or a plant having all physiological and/or morphological characteristics of NUN 70039 PPH or NUN 70041 PPH or NUN 70042 PPH but one, two or three different characteristics), such as a cutting, a cell culture or a tissue culture.

The invention also concerns methods of vegetatively propagating a plant of the invention. In certain embodiments, the method comprises the steps of: (a) collecting tissue or cells capable of being propagated from a plant of the invention; (b) cultivating said tissue or cells to obtain proliferated shoots; and (c) rooting said proliferated shoots, to obtain rooted plantlets. Steps (b) and (c) may also be reversed, i.e. first cultivating said tissue to obtain roots and then cultivating the tissue to obtain shoots, thereby obtaining rooted plantlets. The rooted plantlets may then be further grown, to obtain plants. In one embodiment, the method further comprises step (d) growing plants from said rooted plantlets

The invention also provides for a vegetatively propagated plant of variety NUN 70039 PPH or NUN 70041 PPH or NUN 70042 PPH (or from progeny of said varieties or from or a plant having all but one, two or three physiological and/or morphological characteristics of NUN 70039 PPH or NUN 70041 PPH or NUN 70042 PPH, or a part thereof, having one or more distinguishing characteristics and/or all or all but one, two or three of the morphological and physiological characteristics of NUN 70039 PPH or NUN 70041 PPH or NUN 70042 PPH, when grown under the same environmental conditions.

A part of a variety of the invention, i.e. NUN 70039 PPH or NUN 70041 PPH or NUN 70042 PPH (or of progeny of said varieties or of a plant having all physiological and/or morphological characteristics but one, two or three which are different from those of NUN 70039 PPH or NUN 70041 PPH or NUN 70042 PPH) encompasses any cells, tissues, organs obtainable from the seedlings or plants, such as but not limited to: a pepper fruit or a part thereof, a cutting, hypocotyl, cotyledon, seedcoat, pollen and the like. Such parts can be stored and/or processed further. Encompassed are therefore also food or feed products comprising one or more of such parts, such as canned, chopped, cooked, roasted, in a sauce, in a sandwich, pasted, puréed or concentrated, juiced, frozen, dried, pickled, or powdered pepper fruit from NUN 70039 PPH or NUN 70041 PPH or NUN 70042 PPH or from progeny of said varieties, or from a derived variety, such as a plant having all but one, two or three physiological and/or morphological characteristics of NUN 70039 PPH or NUN 70041 PPH or NUN 70042 PPH.

In one aspect a haploid plant and/or a double haploid plant of a variety of the invention, i.e. NUN 70039 PPH or NUN 70041 PPH or NUN 70042 PPH, or a plant having all but one, two or three physiological and/or morphological characteristics of NUN 70039 PPH or NUN 70041 PPH or NUN 70042 PPH, or progeny of any of these, are encompassed herein. Haploid and double haploid (DH) plants can, for example, be produced by cell or tissue culture and chromosome doubling agents and regeneration into a whole plant. For DH production chromosome doubling may be induced using known methods, such as colchicine treatment or the like.

In yet another aspect haploid plants and/or double haploid plants derived from NUN 70039 PPH that, when combined, make a set of parents of NUN 70039 PPH, or haploid plants and/or double haploid plants derived from NUN 70041 PPH that, when combined, make a set of parents of NUN 70041 PPH, or haploid plants and/or double haploid plants derived from NUN 70042 PPH that, when combined, make a set of parents of NUN 70042 PPH are encompassed herein.

Using methods known in the art like “reverse breeding”, it is possible to produce parental lines for a hybrid plant such as NUN 70039 PPH or NUN 70041 PPH or NUN 70042 PPH; where normally the hybrid is produced from the parental lines. Such methods are based on the segregation of individual alleles in the spores produced by a desired plant and/or in the progeny derived from the self-pollination of that desired plant, and on the subsequent identification of suitable progeny plants in one generation, or in a limited number of inbred cycles. Such a method is known from WO2014076249 or from Nature Protocols Volume: 9, Pages: 761-772 (2014) DOI: doi:10.1038/nprot.2014.049, which are enclosed by reference. Such method for producing parental lines for a hybrid organism, comprises the steps of: a) defining a set of genetic markers that are present in a heterozygous form (H) in a partially heterozygous starting organism; b) producing doubled haploid lines from spores of the starting organism: c) genetically characterizing the doubled haploid lines thus obtained for the said set of genetic markers to determine whether they are present in a first homozygous form (A) or in a second homozygous form (B); d) selecting at least one pair of doubled haploid lines that have complementary alleles for at least a subset of the genetic markers, wherein each member of the pair is suitable as a parental line for a hybrid organism.

Thus in one aspect, the invention relates to a method of producing a combination of parental lines of a plant of the invention (NUN 70039 PPH or NUN 70041 PPH or NUN 70042 PPH) comprising the step of making double haploid cells from haploid cells from the plant of the invention or a seed of that plant; and optionally crossing these parental lines to produce and collect seeds. In another aspect, the invention relates to a combination of parental lines produced by this method. In still another aspect said combination of parental lines can be used to produce a seed or plant of the invention, i.e. NUN 70039 PPH or NUN 70041 PPH or NUN 70042 PPH, when these parental lines are crossed. In still another aspect, the invention relates to a combination of parental lines from which a seed or plant having all physiological and/or morphological characteristics (when grown under the same environmental conditions) of a plant of the invention (NUN 70039 PPH or NUN 70041 PPH or NUN 70042 PPH) but one, two or three which are different can be produced. In still another aspect, the invention relates to a combination of parental lines from which a seed or plant having all the characteristics of or plant of the invention, i.e. NUN 70039 PPH or NUN 70041 PPH or NUN 70042 PPH, as defined in Table 1 and/or 2 when grown under the same conditions can be produced.

In another alternative aspect, the invention provides a method of introducing a single locus conversion or single trait conversion or a desired trait into NUN 70039 PPH or NUN 70041 PPH or NUN 70042 PPH comprising:
a. obtain a combination of a male and a female parental line of NUN 70039 PPH or NUN 70041 PPH or NUN 70042 PPH,
b. introduce a single locus conversion in at least one of the parents of step a;
c. crossing the converted parent with the other parent of step a to obtain seed of NUN 70039 PPH or NUN 70041 PPH or NUN 70042 PPH
   A combination of a male and a female parental line of NUN 70039 PPH or NUN 70041 PPH or NUN 70042 PPH can be generated by methods described herein, for example through reverse breeding.

In an embodiment of the invention, step b) of the above method—introduce a single locus conversion in at least one of the parents of step a—may be done through the following method:
i. obtaining a cell or tissue culture of cells of the parental line of NUN 70039 PPH or NUN 70041 PPH or NUN 70042 PPH;
ii. genetically transforming or mutating said cells;
iii. growing the cells into a plant; and
iv. optionally selecting plants that contain the single locus conversion, the single trait conversion or the desired trait.

In another embodiment of the invention, step b) of the above method—introduce a single locus conversion in at least one of the parents of step a—may be done through the following method:
i. crossing the parental line of NUN 70039 PPH or NUN 70041 PPH or NUN 70042 PPH with a second pepper plant comprising the single locus conversion, the single trait conversion or the desired trait;
ii. selecting F1 progeny plants that contain the single locus conversion, the single trait conversion or the desired trait;
iii. crossing said selected progeny plants of step ii with the parental line of step i, to produce a backcross progeny plant;
iv. selecting backcross progeny plants comprising the single locus conversion, the single trait conversion or the desired trait and otherwise all or all but one, two or three of the morphological and physiological characteristics the parental line of step i to produce selected backcross progeny plants; and
v. optionally repeating steps iii and iv one or more times in succession to produce selected second, third or fourth or higher backcross progeny plants comprising the single locus conversion, the single trait conversion or the desired trait and otherwise all or all but one, two or three of the morphological and physiological characteristics the parental line of step i to produce selected backcross progeny plants, when grown in the same environmental conditions.
   The invention further relates to plants obtained by this method.

The above method is provided, wherein the single locus conversion concerns a trait, wherein the trait is pest resistance or disease resistance. In one embodiment the trait is disease resistance and the resistance is conferred to Cucumber Mosaic Virus, Curly Top Virus, Pepper Mottle Virus, Potato Y Virus, Tobacco Etch Virus, Tobacco Mosaic Virus, Anthracnose (Gloeosporium piperatum), Bacterial Spot (Xanthomonas vesicatoria), Cercospora Leaf Spot (Cercospora capsici), Nematode (Meloidogyne incognita acrita), Phytophthora Root Rot (Phytophthora capsici), Ripe Rot (Vermicularia capsici), Southern Blight (Sclerotium rolfsii) and/or Verticillium Wilt (Verticillium dahliae). Other resistance genes, against pathogenic viruses, fungi, bacteria, nematodes, insects or other pests may also be introduced. In an embodiment, the resistance is TSWV resistance or Ve427^{RB} resistance.

Also provided are plant parts derived from variety NUN 70039 PPH or NUN 70041 PPH or NUN 70042 PPH (or from progeny of said varieties or from a plant having all but one, two or three physiological and/or morphological characteristics which are different from those of NUN 70039 PPH or NUN 70041 PPH or NUN 70042 PPH, or from a vegetatively propagated plant of NUN 70039 PPH or NUN 70041 PPH or NUN 70042 PPH (or from its progeny or from a plant having all but one, two or three physiological and/or morphological characteristics which are different from those of NUN 70039 PPH or NUN 70041 PPH or NUN 70042 PPH, being selected from the group consisting of a fruit, a harvested fruit, a part of a fruit, a leaf, a part of a leaf, pollen, an ovule, a cell, a petiole, a shoot or a part thereof, a stem or a part thereof, a root or a part thereof, a root tip, a cutting, a seed, a part of a seed, seedcoat or another maternal tissue which is part of a seed grown on NUN 70039 PPH or NUN 70041 PPH or NUN 70042 PPH, hypocotyl, cotyledon, a scion, a stock, a rootstock, a pistil, an anther, and a flower or a part thereof.

In one embodiment, the invention provides for extracts of a plant described herein and compositions comprising or consisting of such extracts. In a preferred embodiment, the extract consists of or comprises tissue of a plant described herein or is obtained from such tissue.

In still yet another aspect, the invention provides a method of determining the genotype of a plant of the invention comprising the step of detecting in the genome (e.g., a sample of nucleic acids) of the plant at least a first polymorphism or an allele. The skilled person is familiar with many suitable methods of genotyping, detecting a polymorphism or detecting an allele including restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, and hybridization to DNA microarrays or beads. Alternatively, the entire genome could be sequenced. The method may, in certain embodiments, comprise detecting a plurality of polymorphisms in the genome of the plant, for example by obtaining a sample of nucleic acid from a plant and detecting in said nucleic acids a plurality of polymorphisms. The method may further comprise storing the results of the step of detecting the plurality of polymorphisms on a computer readable medium

The invention also provides for a food or feed product comprising or consisting of a plant part described herein wherein the plant part can be identified as a part of the plant of the invention. Preferably, the plant part is a pepper fruit or part thereof and/or an extract from a fruit or another plant part described herein. The food or feed product may be fresh or processed, e.g., dried, grinded, powdered, pickled, chopped, cooked, roasted, in a sauce, in a sandwich, pasted, puréed or concentrated, juiced, pickled, canned, steamed, boiled, fried, blanched and/or frozen, etc.

For example, containers such as cans, boxes, crates, bags, cartons, Modified Atmosphere Packagings, films (e.g. biodegradable films), etc. comprising a plant or a parts of a plant (fresh and/or processed) described herein or a seed of NUN 70041 PPH are also provided herein.

Marketable pepper fruits are generally sorted by size and quality after harvest. Alternatively the pepper fruits can be sorted by pungency.

Pepper may also be grown for use in grafting or inosculation as rootstocks (stocks) or scions (cions). Typically, different types of pepper are grafted to enhance disease resistance, which is usually conferred by the rootstock, while retaining the horticultural qualities usually conferred by the scion. It is not uncommon for grafting to occur between cultivated pepper varieties and related Solanacea species. Methods of grafting and vegetative propagation are well-known in the art.

So in one aspect the invention relates to a plant comprising a rootstock or scion of NUN 70041 PPH.

All documents (e.g., patent publications) are herein incorporated by reference in their entirety.

### CITED REFERENCES

Tiwari et al., BMC Plant Biology 201111:143 DOI: 10.1186/1471-2229-11-143
WO 2013078319
US patent application no. 20060037100, Feb. 16, 2006
Sang-Gu et al. (1988), Plant Cell, Tissue and Organ Culture 12: 67-74
Kothari et al., (2010) Biotechnology Advances 28: 35-48
“Guidelines for the Conduct of Tests for Distinctness, Uniformity and Stability, TG/76/8 (Geneva, 2006—updated 2015), as published by UPOV (International Union for the Protection of New Varieties and Plants: upov.int/under edocs/tgdocs/en/tg076.pdf
US Department of Agriculture, Agricultural Marketing Service, Plant Variety Protection Office, Beltsville, Md.: ams.usda.gov/sites/default/files/media/56-Pepper.pdf.
Vos et al. 1995, Nucleic Acid Research 23: 4407-4414)
Ince et al., (2010) Biochem. Genet. 48:83-95)
WO2013182646
Thabuis et al., (2004) Theor Appl Genet 109:342-351)
Principles of Plant Genetics and Breeding, 2007, George Acquaah, Blackwell Publishing, ISBN-13: 978-1-4051-3646-4
WO2014076249
Nature Protocols Volume: 9, Pages: 761-772 (2014) DOI: doi:10.1038/nprot.2014.049

### Examples

### Development of NUN 70039 PPH

The hybrid NUN 70039 PPH was developed from a male and female proprietary inbred line of Nunhems. The female and male parents were crossed to produce hybrid (F1) seeds of NUN 70039 PPH The seeds of NUN 70039 PPH can be grown to produce hybrid plants and parts thereof (e.g. pepper fruit). The hybrid NUN 70039 PPH can be propagated by seeds or vegetative.

The hybrid variety is uniform and genetically stable. This has been established through evaluation of horticultural characteristics. Several hybrid seed production events resulted in no observable deviation in genetic stability. Coupled with the confirmation of genetic stability of the female and male parents the Applicant concluded that NUN 70039 PPH is uniform and stable.

### Development of NUN 70041 PPH

The hybrid NUN 70041 PPH was developed from a male and female proprietary inbred line of Nunhems. The female and male parents were crossed to produce hybrid (F1) seeds of NUN 70041 PPH The seeds of NUN 70041 PPH can be grown to produce hybrid plants and parts thereof (e.g. pepper fruit). The hybrid NUN 70041 PPH can be propagated by seeds or vegetative.

The hybrid variety is uniform and genetically stable. This has been established through evaluation of horticultural characteristics. Several hybrid seed production events resulted in no observable deviation in genetic stability. Coupled with the confirmation of genetic stability of the female and male parents the Applicant concluded that NUN 70041 PPH is uniform and stable.

### Development of NUN 70042 PPH

The hybrid NUN 70042 PPH was developed from a male and female proprietary inbred line of Nunhems. The female and male parents were crossed to produce hybrid (F1) seeds of NUN 70042 PPH The seeds of NUN 70042 PPH can be grown to produce hybrid plants and parts thereof (e.g. pepper fruit). The hybrid NUN 70042 PPH can be propagated by seeds or vegetative.

The hybrid variety is uniform and genetically stable. This has been established through evaluation of horticultural characteristics. Several hybrid seed production events resulted in no observable deviation in genetic stability. Coupled with the confirmation of genetic stability of the female and male parents the Applicant concluded that NUN 70042 PPH is uniform and stable.

### Deposit Information

A total of 2500 seeds of the hybrid variety NUN 70039 PPH are deposited according to the Budapest Treaty by Nunhems B.V. on ______, at or at the NCIMB Ltd., Ferguson Building, Craibstone Estate, Bucksburn, Aberdeen AB21 9YA, United Kingdom (NCIMB). The deposit has NCIMB number ______. A deposit of NUN 70039 PPH and of the male and female parent line is also maintained at Nunhems B.V.

A total of 2500 seeds of the hybrid variety NUN 70041 PPH are deposited according to the Budapest Treaty by Nunhems B.V. on ______, at or at the NCIMB Ltd., Ferguson Building, Craibstone Estate, Bucksburn, Aberdeen AB21 9YA, United Kingdom (NCIMB). The deposit has NCIMB number ______. A deposit of NUN 70041 PPH and of the male and female parent line is also maintained at Nunhems B.V.

A total of 2500 seeds of the hybrid variety NUN 70042 PPH are deposited according to the Budapest Treaty by Nunhems B.V. on ______, at or at the NCIMB Ltd., Ferguson Building, Craibstone Estate, Bucksburn, Aberdeen AB21 9YA, United Kingdom (NCIMB). The deposit has NCIMB number ______. A deposit of NUN 70042 PPH and of the male and female parent line is also maintained at Nunhems B.V.

Access to the deposits will be available during the pendency of this application to persons determined by the Director of the U.S. Patent Office to be entitled thereto upon request. Subject to 37 C.F.R. § 1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 USC 2321 et seq.).

The most similar variety to NUN 70039 PPH is Piton, a variety from US Agriseeds. In Table 1 a comparison between NUN 70039 PPH and Piton is shown based on a trial in the USA. The most similar variety to NUN 70041 PPH is Piton, a variety from US Agriseeds. Table 1 also shows a comparison between NUN 70041 PPH and Piton is shown based on a trial in the USA. The most similar variety to NUN 70042 PPH is Forajido, a variety from ENZA. In Table 1 a comparison between NUN 70042 PPH and Forajudo is shown based on a trial in the USA. Trial location Acampo, Calif. 38192873 N, 121.232637W. transplanting date for NUN 70041 PPH: 19 Sep. 2016.

Two replications of 50 plants each, from which 15 plants or plant parts were randomly selected, were used to measure characteristics. In Table 1 the USDA descriptors of NUN 70039 PPH and NUN 70041 PPH (this application) and REFERENCE VARIETY Piton are listed, as well as the USDA descriptors of NUN 70042 PPH (this application) and REFERENCE VARIETY Forajido.

In accordance with one aspect of the present invention, there is provided a plant having the physiological and morphological characteristics of pepper varieties NUN 70039 PPH or NUN 70041 PPH or NUN 70042 PPH as presented in Table 1.

TABLE 1
USDA descriptors of varieties NUN 70039 PPH and NUN 70041 PPH, which both have REFERENCE
VARIETY Piton, as well as NUN 70042 PPH and its REFERENCE VARIETY Forajido

| | Application | Application | Reference | Application | Reference |
|---|---|---|---|---|---|
| | variety | variety | variety | variety | variety |
| USDA descriptor | NUN 70039 PPH | NUN 70041 PPH | Piton | NUN 70042 PPH | Forajido |
| 1. SPECIES: 1 = C. annuum 2 = C. frutescens 3 = C. | 1 | 1 | 1 | 1 | 1 |
| baccatum 4 = C. chinense 5 = Other (specify) | | | | | |
| 2. MATURITY | | | | | |
| (In Region of Best Adaptability): | | | | | |
| Days from transplanting until mature green stage | 80 | 80 | 80 | n.r. | n.r. |
| Days from transplanting until mature red or yellow stage | n.r. | n.r. | n.r. | n.r. | n.r. |
| Days from direct seeding until mature green stage | n.r. | n.r. | n.r. | n.r. | n.r. |
| Days from direct seeding until mature red or yellow | n.r. | n.r. | n.r. | n.r. | n.r. |
| stage | | | | | |
| 3. PLANT | | | | | |
| Plant Habit: 1 = Compact 2 = Semi-spreading | 2 | 2 | 2 | 2 | 2 |
| 3 = Spreading 4 = Other | | | | | |
| Plant Attitude : 1 = Erect 2 = Semi-erect 3 = Prostrate | 1 | 1 | 1 | 1 | 1 |
| 4 = Other | | | | | |
| Plant Height (cm) | 55.4 | 55.4 | 58.9 | 60.0 | 55.2 |
| Plant Width (cm) | 64.4 | 60.0 | 58.9 | 49.8 | 64.5 |
| Length of Stem from Cotyledons to First Flower (cm) | 22.3 | 19.3 | 20.5 | 17.5 | 17.3 |
| Length of Third Internode (from soil surface) (mm) | 32.8 | 39.7 | 38.0 | 32.8 | 28.9 |
| Basal Branches: 1 = None 2 = Few (2-3) 3 = Many (more | 1 | 1 | 1 | 1 | 1 |
| than 3) | | | | | |
| Branch Flexibility: 1 = Willowy (Cayenne Long Red) | 2 | 2 | 2 | 2 | 2 |
| 2 = Rigid (Yolo Wonder L) | | | | | |
| Stem Strength (Breakage Resistance): | 3 | 3 | 3 | 3 | 3 |
| 1 = Weak 2 = Intermediate 3 = Strong | | | | | |
| 4. LEAVES: | | | | | |
| Leaf Width (mm) | 65.0 | 64.0 | 65.6 | 46.7 | 67.1 |
| Leaf Length (mm) | 126.1 | 101.5 | 126.0 | 109.4 | 155.8 |
| Petiole Length (mm) | 72.7 | 70.8 | 54.4 | 54.4 | 83.4 |
| Mature Leaf Shape: 1 = Lanceolate 2 = Elliptic | 2 | 2 | 2 | 2 | 2 |
| Leaf Color: 1 = Light Green 2 = Medium Green 3 = Dark | 2 | 2 | 2 | 2 | 2 |
| Green 4 = Purple 5 = Other (specify) | | | | | |
| Color Chart Name Code: RHS | Green N137A | Green N137A | Green N137B | Green N137B | Green N137A |
| Leaf and Stem Pubescence: 1 = Absent (Yolo Wonder L) | 1 | 1 | 1 | 1 | 1 |
| 2 = Light 3 = Moderate (Serrano) 4 = Heavy (Chili Piquin) | | | | | |
| Margin Undulation: 1 = Absent 2 = V. Weak 3 = Weak | 2 | 2 | 2 | 2 | 2 |
| 4 = Medium 5 = Strong 6 = V. Strong | | | | | |
| Blistering: 1 = Absent 2 = Very Weak 3 = Weak 4 = | 3 | 1 | 3 | 2 | 3 |
| Medium 5 = Strong 6 = Very Strong | | | | | |
| 5. FLOWERS: | | | | | |
| Number of Flowers per Leaf Axil | 1.0 | 1.1 | 1.1 | 1 | 1.1 |
| Number of Calyx Lobes | 6.9 | 6.6 | 6.4 | 6.3 | 6.9 |
| Number of Petals | 6.5 | 6.0 | 6.2 | 6.4 | 6.1 |
| Flower Diameter (mm) | 25.2 | 27.7 | 25.1 | 22.7 | 24.6 |
| Corolla Color: 1 = White 2 = Purple 3 = Other (Specify) | 1 | 1 | 1 | 1 | 1 |
| Corolla Throat Markings: 1 = Yellow (Tan) 2 = Purple | 1 | 1 | 1 | 1 | 1 |
| 3 = Other (Specify) | | | | | |
| Anther Color: 1 = Yellow 2 = Purple 3 = Other (Specify) | 2 | 2 | 2 | 2 | 2 |
| Style Length: 1 = Less Than Stamen 2 = Same as Stamen | 2 | 2 | 2 | 3 | 2 |
| 3 = Exceeds Stamen | | | | | |
| Self-Incompatibility: 1 = Absent 2 = Present | n.r. | n.r. | n.r. | n.r. | n.r. |
| 6. FRUIT: | | | | | |
| Group: 1 = Bell (Yolo Wonder L) 2 = Pimiento (Pimiento | 7 | 7 | 7 | 7 | 7 |
| Perfection) 3 = Ancho (Mexican Chili) 4 = Anaheim Chili | | | | | |
| (Sandia) 5 = Cayenne (Cayenne Long Red) 6 = Cuban | | | | | |
| (Cubanelle) 7 = Jalapeno (Jalapeno) 8 = Small Hot | | | | | |
| (Serrano) 9 = Cherry (Sweet Cherry) 10 = Short Wax | | | | | |
| (Floral Gem) 11 = Long Wax (Sweet Banana) | | | | | |
| 12 = Tabasco (Tabasco) 13 = Habanero (Scotch Bonnet) | | | | | |
| 14 = Other | | | | | |
| Immature Fruit Color: 1 = Light Green (Cubanelle) | 3 | 3 | 3 | 3 | 2 |
| 2 = Medium Green (Long Thin Cayenne) 3 = Dark Green | | | | | |
| (Yolo Wonder L) 4 = Very Dark Green (Ancho Chili) | | | | | |
| 5 = Yellow (Yellow Belle) 6 = Purple (Violetta) 7 = Ivory | | | | | |
| (Twiggy) 8 = Other | | | | | |
| Immature Fruit Color: Color Chart Name Code: RHS | Green 139A | Green 137A | Green 137A | Green 139A | Green 137A |
| Mature Fruit Color: 1 = Red (Yolo Wonder L) 2 = Orange | 1 | 1 | 1 | 1 | 1 |
| 3 = Orange-Yellow (Golden Calwonder) 4 = Brown | | | | | |
| (Mulatto) 5 = Ivory 6 = Green (Permagreen) 7 = Salmon | | | | | |
| 8 = Lemon Yellow 9 = Other, | | | | | |
| Mature Fruit Color: Color Chart Name Code: RHS | n.r. | n.r. | n.r. | n.r. | n.r. |
| Pungency: 1 = Sweet (Yolo Wonder L) 2 = Hot (Jalapeno) | 2 | 2 | 2 | 2 | 2 |
| mg Capsaicin per gram dry fruit | n.r. | n.r. | n.r. | n.r. | n.r. |
| Scoville Units (dry fruit) | n.r. | n.r. | n.r. | n.r. | n.r. |
| Flavor: 1 = Mild Pepper Flavor 2 = Moderate Pepper | 2 | 2 | 2 | 2 | 2 |
| Flavor 3 = Strong Pepper Flavor 4 = Other, | | | | | |
| Fruit Glossiness: 1 = Dull 2 = Moderate 3 = Shiny | 3 | 3 | 3 | 3 | 3 |
| Surface Smoothness: 1 = Smooth (Yolo Wonder L) | 1 | 1 | 1 | 1 | 1 |
| 2 = Rough (Long Thin Cayenne) | | | | | |
| Fruit Position: 1 = Upright (Santaka) 2 = Horizontal | 3 | 3 | 3 | 3 | 3 |
| 3 = Pendent (Jalapeno) | | | | | |
| Calyx Shape: 1 = Cup-shaped (Enveloping Fruit Base) | 2 | 2 | 2 | 2 | 2 |
| 2 = Saucer-shaped (Flat, Non-Enveloping) | | | | | |
| Calyx Diameter (mm) | 19.0 | 20.5 | 21.5 | 18.1 | 19.4 |
| Fruit Length (mm) | 114.2 | 107.9 | 116.4 | 99.5 | 83.0 |
| Fruit Diameter at Calyx Attachment (mm) | 31.4 | 32 | 31.2 | 27.2 | 24.6 |
| Fruit Diameter at Mid-point (mm) | 32.6 | 33.5 | 35.5 | 29.1 | 29.0 |
| Flesh Thickness at Mid-point (mm) | 4.8 | 5.0 | 4.3 | 4.1 | 4.1 |
| Average Number of Fruits per Plant | n.r. | n.r. | n.r. | n.r. | n.r. |
| % Large fruits | n.r. | n.r. | n.r. | n.r. | n.r. |
| % Medium fruits | n.r. | n.r. | n.r. | n.r. | n.r. |
| % small fruits | n.r. | n.r. | n.r. | n.r. | n.r. |
| Average Fruit Weight (gm) | 53.3 | 53.9 | 52.8 | 36.5 | 33.5 |
| Fruit Base Shape: 1 = Cupped (Yolo Wond L), | 2 | 2 | 2 | 2 | 2 |
| 2 = Rounded (Jalapeno) | | | | | |
| Fruit Apex Shape: 1 = Pointed (Long Thin Cayenne) | 1 | 1 | 1 | 1 | 2 |
| 2 = Blunt (Yolo Wonder L) | | | | | |
| Fruit Shape: 1 = Bell (Yolo Wonder L) 2 = Conical | 4 | 4 | 4 | 4 | 4 |
| (Pimiento) 3 = Elongate (Long Thin Cayenne) 4 = Oblong | | | | | |
| (Jalapeno) 5 = Oblate (Sunnybrook) 6 = Globe (Red | | | | | |
| Cherry) 7 = Other | | | | | |
| Fruit Shape (Longitudinal Section, see attached | 6 | 6 | 6 | 6 | 6 |
| pictures): 1 = Flattened 2 = Round 3 = Heart-shaped | | | | | |
| 4 = Square 5 = Rectangular 6 = Trapezoid 7 = Narrow | | | | | |
| Triangular 8 = Triangular 9 = Horn-shaped | | | | | |
| Fruit Shape (Cross Section, at Level of Placenta): | 4 | 4 | 4 | 4 | 4 |
| 1 = Elliptic 2 = Triangular 3 = Quadrangular 4 = Circular | | | | | |
| Fruit Set: 1 = Scattered 2 = Concentrated | 2 | 2 | 2 | 2 | 2 |
| Interloculary Grooves: 1 = Absent 2 = Very Shallow | 1 | 1 | 1 | 1 | 1 |
| 3 = Shallow 4 = Medium 5 = Deep 6 = Very Deep | | | | | |
| % Fruits with one locule | 0 | 0 | 0 | 0 | 0 |
| % Fruits with two locules | 26.67 | 6.67 | 0 | 6.67 | 0 |
| % Fruits with three locules | 53.3 | 60.0 | 53.3 | 60.0 | 73.3 |
| % Fruits with four locules | 13.33 | 33.33 | 40.0 | 40.0 | 26.7 |
| % Fruits with five or more locules | 6.67 | 0 | 6.67 | 0 | 0 |
| Average Number of Locules | 3.0 | 3.3 | 3.53 | 3.3 | 3.3 |
| Pedicel Length (mm) | 32.2 | 30.0 | 23.6 | 30.4 | 31.7 |
| Pedicel Thickness (mm) | 4.9 | 5.12 | 5.0 | 4.2 | 5.2 |
| Pedicel Shape: 1 = Straight 2 = Curved | 2 | 2 | 2 | 2 | 2 |
| Pedicel Cavity: 1 = Absent 2 = Present | 1 | 1 | 1 | 1 | 1 |
| 7 SEED: | | | | | |
| Seed Cavity Length (mm) | 102.6 | 97.9 | 104 | 92.2 | |
| Seed Cavity Diameter (mm) | 29.9 | 30.71 | 29.2 | 25.9 | |
| Placenta Length (mm) | 47.5 | 53.5 | 54.0 | 51.6 | |
| Number of Seeds per Fruit | 156 | 146.7 | 169 | 164.7 | |
| Gm per 1000 seeds | n.r. | n.r. | n.r. | n.r. | n.r. |
| Seed Color 1 = Yellow; 2 = Purple | 1 | 1 | 1 | 1 | 1 |
| 8. ANTHOCYANIN (1 = Absent; 2 = Weak; 3 = | | | | | |
| Moderate; 4 = Strong): | | | | | |
| Seedling Hypocotyl | 1 | 1 | 1 | n.r. | n.r. |
| Stem | 2 | 2 | 2 | n.r. | n.r. |
| Node | 3 | 3 | 3 | n.r. | n.r. |
| Leaf | 1 | 1 | 1 | n.r. | n.r. |
| Pedicel | 1 | 1 | 1 | n.r. | n.r. |
| Calyx | 1 | 1 | 1 | n.r. | n.r. |
| Fruit | 1 | 1 | 1 | n.r. | n.r. |

TABLE 2
Non-USDA descriptors of varieties NUN 70039 PPH and NUN 70041 PPH, which both have REFERENCE
VARIETY Piton, as well as NUN 70042 PPH and its REFERENCE VARIETY Forajido

| | Application | Application | Reference | Application | Reference |
|---|---|---|---|---|---|
| | variety | variety | variety | variety | variety |
| Non-USDA descriptor | NUN 70039 PPH | NUN 70041 PPH | Piton | NUN 70042 PPH | Forajido |
| Petiole Length (mm) | 3.1 | 2.7 | 3.3 | 2.8 | 3.6 |

Table 1 and 2 contain typical values. Values may vary due to environment. Other values that are substantially equivalent are also within the scope of the invention. N.A.=not applicable; n.r.=not recorded.

1. A plant, plant part or seed of pepper variety NUN 70042 PPH, wherein a representative sample of said seed has been deposited under Accession Number NCIMB ______.

2. The plant part of claim 1, wherein said plant part is a leaf, pollen, an ovule, a fruit, a scion, a rootstock, cutting, flower or a part of any of these or a cell.

3. A seed grown on the plant of claim 1.

4. A Capsicum plant, or a part thereof which does not significantly differ from the plant of claim 1 in any of the 10 distinguishing characteristics: 1) average fruit length; 2) average fruit width; 3) typical fruit apex shape; 4) average pedicel thickness; 5) average fruit seed cavity length; 6) average fruit seed cavity diameter; 7) typical leaf blistering; 8) typical style length; 9) average leaf length; and 10) average plant width when grown under the same conditions.

5. A pepper plant, or a part thereof which does not significantly differ from the plant of claim 1 when grown under the same conditions.

6. A tissue or cell culture of regenerable cells of the plant of claim 1.

7. The tissue or cell culture according to claim 6, comprising cells or protoplasts from a plant part, wherein the plant part is an embryo, a meristem, a cotyledon, a hypocotyl, pollen, a leaf, an anther, a root, a root tip, a pistil, a petiole, a flower, a fruit, seed, a stem or a stalk.

8. A pepper plant regenerated from the tissue or cell culture of claim 6, wherein the plant has all of the physiological and morphological characteristics of the plant of NUN 70042 PPH, wherein a representative sample of seed of said variety has been deposited under Accession Number NCIMB ______ as listed in Table 1 and/or 2 when determined at the 5% significance level for plants grown under the same conditions.

9. A method of producing the plant of claim 1, or a part thereof, comprising vegetative propagation of the plant of claim 1.

10. The method of claim 9, wherein said vegetative propagation comprises regenerating a whole plant from a part of the plant.

11. The method of claim 9, wherein said part is a cutting, a cell culture or a tissue culture.

12. A vegetative propagated plant of claim 1, or a part thereof, wherein the plant has all of the physiological and morphological characteristics of the plant of claim 1 determined at the 5% significance level for plants grown under the same conditions.

13. A method of producing a pepper plant, comprising crossing the plant of claim 1 with a second pepper plant one or more times, and selecting progeny from said crossing and optionally allowing the progeny to form seed.

14. A progeny plant of the plant of claim 1 obtained by further breeding with said variety.

15. A pepper plant having one, two or three physiological and/or morphological characteristics which are different from those of the plant of claim 1 and which otherwise has all the physiological and morphological characteristics of the plant of claim 1 as listed in Table 1 and/or 2, determined at the 5% significance level for plants grown under the same conditions.

16. A food or feed product comprising the plant part of claim 2.

17. A pepper plant comprising at least a first set of the chromosomes of the plant of claim 1.

18. The plant of claim 1, further comprising a single locus conversion, wherein said plant has all or all but one, two or three of the morphological and physiological characteristics of the plant of claim 1 when grown under the same conditions, optionally wherein the single locus conversion confers of male sterility, herbicide tolerance, insect resistance, pest resistance, disease resistance, environmental stress tolerance, modified carbohydrate metabolism or modified protein metabolism.

19. A plant comprising the scion or rootstock of claim 2.

20. A method of producing a combination of parental lines of the plant of claim 1 comprising making double haploid cells from haploid cells of the plant of claim 1 or a seed from the plant of claim 1.

21. A combination of two inbred plants which when crossed produces a seed or plant of variety NUN 70042 PPH, wherein a representative sample of seed of said variety has been deposited under Accession Number NCIMB ______.

22. A container comprising a plant, plant part or seed of the plant of claim 1.

23. A plant or a cell thereof produced in a method of producing a plant having a desired trait, wherein the method comprises mutating a plant of variety NUN 70042 PPH and selecting a plant with the desired trait, wherein the mutated plant retains all or all but one of the phenotypic and morphological characteristics of variety NUN 70042 PPH as described in Table 1 and contains the desired trait, and wherein a representative sample of seed of variety NUN 70042 PPH has been deposited under Accession Number NCIMB ______.

24. The plant or cell of claim 23, wherein the desired trait is fruit quality, male sterility, herbicide tolerance, insect resistance, pest resistance, disease resistance, virus resistance, TSWV resistance, Ve427^{RB} resistance, environmental stress tolerance, modified carbohydrate metabolism, modified protein metabolism or ripening.
